(19) United States
(12) Patent Application Publication
(10) Pub. No.: US 2006/0292162 A1
(43) Pub. Date: Dec. 28, 2006

(54) Plasma or serum fraction for the treatment or prevention of bacterial infections

(76) Inventor: Robert W. Buckheit JR., Myersville, MD (US)

Correspondence Address: KING & SPALDING LLP, 1180 PEACHTREE STREET, ATLANTA, GA, 30309, US

(21) Appl. No.: 11/410,493

(22) Filed: Apr. 24, 2006

Related U.S. Application Data
(60) Provisional application No. 60/674,049, filed on Apr. 22, 2005.

(51) Int. Cl. A61K 39/40; C12P 21/04

(52) U.S. Cl. 424/164.1; 435/70.21

(57) ABSTRACT

The present invention relates to a plasma or serum fraction derived from a mammal exposed to an inoculant (e.g., a bacteria-bearing inoculant), which fraction has been depleted of one or more high molecular weight proteins or biological agents present in the unprocessed plasma or serum, as well as to a method to treat and/or prevent bacterial infection with the plasma or serum fraction.

### CROSS REFERENCE TO RELATED APPLICATIONS

The present application claims priority to U.S. Provisional Patent Application No. 60/674,049 filed Apr. 22, 2005

### FIELD OF THE INVENTION

The present invention provides compositions, methods and uses for treating and preventing bacterial infections, including Gram-positive and Gram-negative bacterial infections. The invention also extends to methods of producing the compositions of the present invention

### BACKGROUND OF THE INVENTION

Infectious disease is the leading cause of death worldwide and the third leading cause of death in the United States. Once considered to be virtually eliminated as a public health threat, infectious disease has made a comeback in the last forty years. Many well-known diseases previously controlled (e.g., tuberculosis, malaria, and cholera) have re-emerged since the mid-1970's, often in more virulent and drug-resistant forms. In that same time period, many novel disease agents have been identified, (e.g., including HIV, Ebola, SARS-coV) for which no cures are available. Any number of factors are responsible for the re-emergence of infectious disease, including societal changes (i.e., globalization of the food supply, increases in international travel) and changes in healthcare (i.e., the widespread use of antibiotics). While the underdeveloped world is at particular risk, annual infectious disease-related death rates in the United States have nearly doubled to some 170,000 annually after reaching an historic low in 1980. The resulting costs to the public health and economy are significant.

Bacteria have contributed significantly to the rise in infectious disease. Bacteria as a class include a huge number of organisms, generally classified by biochemical characteristic (i.e., Gram stain characteristics) or morphological characteristics (e.g., bacilli, cocci). They are classified taxonomically as to genera and species, although changes in the names and regroupings over time have caused some confusion in the literature. Many bacteria are harmless to humans while others cause disease only opportunistically. A subset of bacteria are highly pathogenic.

While bacterial infections on the rise, they have become increasingly difficult to treat. Antibiotics, the tradition therapy of choice for bacterial infections, have proven less agile bacterial counterparts and antibiotic resistance has become a significant problem. Not only does antibiotic resistance adversely affect the public health, it carries a significant economic burden, with estimates of the annual cost of treating antibiotic resistant infections in the United States reaching as high as $30 billion (National Institute of Health Fact Sheet: Antibiotic Resistance 2002). There is a clear need for novel therapeutics for the treatment of bacterial infections.

Bacteria can be classified biochemically as either Gram-positive or Gram-negative, based on uptake (either positive or negative) of Gram's stain. The different response relates primarily to the cell-wall structure, wherein the thick peptidoglycan layer characteristic of Gram-positive bacterium retains the methyl violet state of the Gram stain after elution with an organic solvent, whereas the thinner wall of the Gram-negative bacterium does not.

Gram Positive Bacteria

While Gram-positive bacteria are a diverse group, the most clinically important gram positive pathogens include Staphylococcus, Streptococcus and Entercocci. Most common are the Staphylococcus, characterized as spherical bacteria that normally inhabits the skin and mucous membranes of humans and warm-blooded animals. For a comprehensive review of the Staphylococcus and their clinical significance see Kloos, W. B., and T. L. Bannerman. 1995. Staphylococcus and Micrococcus, p. 282-298. In P. R. Murray, E. J. Baron, M. A. Pfaller, F. C. Tenover, and R. H. Yolken (ed.), Manual of clinical microbiology, 6th ed. American Society for Microbiology, Washington, D.C. Some Staphylococci are opportunistic pathogens in humans, causing significant morbidity and mortality. Although Staphylococcus has 32 species, the three most relevant clinical species are Staphylococcus aureus, Staphylococcus epidermidis, and Staphylococcus saprophyticus (and their various serotypes).

Staphylococcus aureus is the most virulent Staphylococcus species and causes a variety of infections in humans, both localized and systemic. Most commonly, S. aureus causes a localized skin infection after a skin break or wound. Another common site of entry is the respiratory system, and Staphylococcal pneumonia is a frequent complication of influenza. More serious illnesses may result when S. aureus enters the blood stream, including osteomyelitis, endocarditis, sepsis and meningitis, among others. S. aureus is a major cause of noscomial (hospital-acquired) infections. Like S. aureus, S. epidermis is also a common cause of noscomial infections. S. epidermidis is particularly associated with infection in patients whose treatments require placement of foreign objects (e.g., shunts or catheters) into the body. S. epidermidis is implicated in neonatal nosocomial sepsis and commonly associated with post-operative wound infections and peritonitis in patients with ambulatory peritoneal dialysis. S. saprophyticus is typically associated with urinary tract infections.

The family Streptococcaceae and related organisms are found in the mouths and 5 intestinal tracts of humans. Some of the taxa are virulent pathogens, causing pharyngitis, respiratory infections, skin and soft tissue infections, dental caries, infective endocarditis, and septicemia. Pathogenic species in humans include S. pyogenes (Group A), S. agalactiae (Group B), S. mutans (viridans), and S. pneumoniae. S. faecalis, or Group D Strep, was recently reclassified into another genus, Enterococcus, as described below.

Streptococcus pyogenes is the etiologic agent responsible for many conditions related to streptococcal infections. It is more commonly known as Group A strep and has more than 6,000 recognized subtypes, with new strains constantly arising. The most common form of infection involves a colonization of the pharyrix known as strep throat. It may also produce sinusitis, otitis, arthritis and bone infections. Subtypes of S. pyogenes can lead to scarlet fever. Some strains prefer skin, producing either superficial (impetigo) or deep (cellulitis) infections. Invasive, toxigenic infections can result in necrotizing fasciitis, myositis and streptococcal toxic shock syndrome. Patients may also develop immune-mediated post-streptococcal sequelae, such as acute rheumatic fever and acute glomerulonephritis.

Streptococcus pneumoniae is a bacterium found in the nose and throat. It is responsible for most cases of community acquired pneumonia. S. pneumoniae is the most common cause of ear infections (otitis media), sepsis (blood infection) in children and is the major cause of bacterial pneumonia in adults (also known as strep pneumonia or pneumococcal pneumonia). S. agalactiae, otherwise known as Group B strep, has emerged as the leading cause of illness and death among newborn infants (Schuchat, A. “Epidemiology of group B streptococcal disease in the United States: shifting paradigms” Clin Microbiol Rev 1998 11:497-513). S. agalactiae, part of the mother's normal microflora, can infect the newborn during delivery and lead to bacteremia, sepsis and meningitis. S. mutans is the etiologic agent of dental caries, one of the most common diseases in humans.

The Enterococci family can be found as part of the normal flora of the gastrointestinal 30 tracts of humans and other animals. Over the last thirty years, Enterococci have become major nosocomial pathogens. They can cause infections at a wide variety of sites, including the urinary tract, bloodstream, endocardium, abdomen, and biliary tract, as well as burn wounds and indwelling devices (Jett B D, Huycke M M, and Gilmore M S. “Virulence of enterococci” Clin. Microbiol. Rev. 1994 7:462-478). Among the 15 species of Enterococci, E faecalis and E faecium are the most commonly associated with clinical infection. E. faecalis was previously known as Streptococcus faecali. Along with other members of the D group, E. faecalis can produce urinary tract infections and endocarditis.

The Gram-positive bacteria Mycobacterium tuberculosis causes tuberculosis (TB) in humans and kills more people each year globally than any other infectious disease. Nearly one-third of the global population is infected with TB. The bacteria can infect any part of the body, but usually attacks the lungs. Most people infected with TB bacteria can successfully overcome infection (“latent TB”), but the bacteria remain alive and can become active later, taking advantage of weaknesses in the immune system. Other people, particularly those with weak immune systems (i.e., infants) develop active TB almost immediately. Symptoms include weakness, weight loss, fever, chills, and sweating. People infected with HIV are particularly at risk, as evidenced by significant increases in TB infection rates in the United States beginning in the mid 1980's (Centers for Disease Control and Prevention: Initial therapy for tuberculosis in the era of multidrug resistance: Recommendations of the Advisory Council for the Elimination of Tuberculosis. mMWR 42(RR-7): 1-8, 1993).

Gram-positive bacteria cause disease through a combination of mechanisms. Colonization, penetration, spread and damage mechanisms are complex. Gram-positive bacteria employ specific disease-causing exotoxins more commonly than Gram-negative bacteria. These toxins are released from intact bacteria and can be present when bacteria are absent (Iandolo, J. J. “Genetic analysis of extracellular toxins of Staphylococcus aureus” Annu. Rev. Microbiol. 1989 25 43:375-402). Some Gram positive species like S. aureus have a complex repertoire of disease causing mechanisms, which are part of the response to environmental stress.

There has been a shift to an increase in number and severity of Gram-positive infections in the last decades (Berger-Bachi B. “Resistance mechanisms of gram-positive bacteria” Int J Med Microbiol. 2002 292(1):27-35). Hospital (noscomial) infections have shifted from Gram-negative organisms to Gram-positive isolates, which now represent the majority of such infections (Ryback M J. “Therapeutic options for Gram positive infections” J Hosp Infec. 2001 49 (Suppl.A): S25-32). The prevalence of sepsis due to Gram-positive bacteria has risen remarkably over the past two decades, and those microorganisms may well predominate as the cause of sepsis within the next few years (Schaberg, et al., Am. J. Med., 1991 91:72S-75S). Overall, Gram-positive bacteria present a global public health threat.

While bacterial infections on the rise, they have become increasingly difficult to treat. Antibiotics have been the therapy of choice for bacterial infections since the 1940s. Yet, antibiotics have proven less agile that their bacterial counterparts and antibiotic resistance has become a significant problem (Stoeckle & Douglas JAMA 1996 275:1816-1817; Beardsley Scientific American 1996 274:26). Multiple surveillance studies have demonstrated that resistance among prevalent pathogens is increasing at a distressing pace, leading to greater morbidity and mortality from nosocomial infections (Jones R N. “Resistance patterns among nosocomial pathogens: trends over the past few years” Chest. 2001 119(2 Suppl):397S-404S). The three most clinically relevant drug resistant Gram-positive bacteria are Staphylococcus aureus (MRSA), penicillin-resistant Streptococcus pneumonia (PRSP) and vancomycine-resistant enterococci (VRE).

Gram positive bacteria accumulate multiple resistances under antibiotic pressure and selection (Berger-Bachi B. “Resistance mechanisms of gram-positive bacteria” Int J Med Microbiol. 2002 292(1):27-35). Overuse of antibiotics, failure to fully complete a full course of antibiotic treatment, routine prophylactic use and sub-therapeutic drug levels all contribute to the development of resistant strains of bacteria (Korff A, Larson E, Kumar P, Peters S, and Jackson D. “Vancomycin-Resistant Enterococcus in a Hospital-Based Dialysis Unit” ANNA Journal. 1998 25(4):381-385).

Antibiotic resistance is a particular problem with Staphylococcus. In 1941, for example, 25 nearly all strains of S. aureus were susceptible to penicillin. By the mid-1940's however, resistance began to emerge. It was not until 1960 that a new antibiotic, methicillin, was developed to combat penicillin-resistant Staphylococcus (PRSA) infections. Today, more than 34% of S. aureus of clinical isolates in the U.S. are resistant to methicillin, 26% in Europe, and 45% in the western Pacific (Diekema D J, Jones R N. “Oxazolidione antibiotics” Lancet 2001 358:1975-1982). Methicillin-resistant S. aureus isolates (MRSA) are particularly common in tertiary care and large city hospitals. These organisms are usually resistant to other antimicrobial agents, including aminoglycosides, chloramphenicol, clindamycin, fluoroquinolones and macrolides. For these resistant strains, vancomycin is the only antibiotic option. Recently, the emergence of MRSA with reduced vancomycin susceptibility suggests that totally resistant S. aureus may soon emerge (Hiramatsu K, Hanaki H, Ino T, Yabuta K, Oguri T, Tenover F C. “Methicillin-resistant Staphylococcus aureus clinical strain with reduced vancomycin susceptibility” J Antimicrob Chemother 1997 40:135-136).

Antibiotic resistance is also a major problem for S. pneumoniae. In the past, S. pneumoniae was almost uniformly susceptible to penicillin. Since the 1960s however, resistance to penicillin and other antimicrobial agents has spread rapidly. In some areas, as many as 30% of isolates are penicillin-resistant Streptococcus pneumoniae (PRSP). The problem of multi-resistance has also spread to PRSP, with isolates that are partially or totally resistant to multiple antibiotics.

Enterococcii are among the most antibiotic resistant human pathogens (Cetinkay Y. “Vancomycin-resistant enterococci” Clinical Microbiology Reviews 2000 13(4):686-707). The acquisition of resistance to vancomycin (VRE) in particular has been on the increase since the late 1980s (Murray B E. “The life and times of the Enterococcus” Clin. Microbiol. Rev. 1990 3:46-65) (Woodford N A, Johnson P, Morrison D, Speller D C E. “Current perspectives on glycopeptide resistance” Clin. Microbiol. Rev. 1995 8:585-615). Vancomycin-resistance is particularly problematic because it frequently occurs in strains that are highly resistant to penicillins and aminoglycosides-a combination with devastating therapeutic consequences (Murray B E. “Vancomycin-resistant enterococci” Am J Med 1997 102(3): 284-93; Spera R V Jr, Farber B F. “Multidrug-resistant Enterococcus faecium. An untreatable nosocomial pathogen” Drugs. 1994 48(5):678-88). Until 1988, vancomycin-resistance was unknown, but increased 20-fold increase over the next several years (Centers for Disease Control and Prevention. 1993. Nosocomial enterococci resistant to vancomycin in the United States 1989-1993. Morbid. Mortal. Weekly Rep. 42:597-599). Studies calculate a mortality rate of up to 37% from enterococcal bacteremia.

There is a need for new drugs to treat Mycobacterium tuberculosis based on the increased frequency of resistance to first- and second-line drugs (i.e. multi-drug resistant TB or MDR-TB) and on the side-effect profiles of currently available drugs (Irwin L L. “New, Unfamiliar Drugs for Suspected Drug-Resistant Tuberculosis” Infect Med 1995 12(1):32-39).

Collectively, adversely affect the public health and creates a significant economic burden—estimates reaching as high as $30 billion annually (National Institute of Health Fact Sheet: Antibiotic Resistance (2002). As physicians and public health agencies struggle to control the factors contributing to resistance, the pharmaceutical industry has focused on the development of new antibiotics active against resistant pathogens (Hunter P. “Growing Threat of Gram-positive resistance—a challenge to the industry” DDT 1997 2: 47-49).

A number of other new antibiotics are in development for Gram-positive infections (Strahilevitz J, Rubinstein E. “Novel agents for resistant Gram-positive infections—a review “Int J Infect Dis. 2002 6 Suppi 1:S38-46; Abbanat D, Macielag M, Bush K “Novel antibacterial agents for the treatment of serious Gram-positive infections” Expert Opin Investig Drugs. 2003 12(3):379-99; Bassetti M, Melica G, Di Biagio A, Rosso R, Gatti G, Bassetti D. “Gram-positive bacterial resistance: future treatment options” Curr Opin Investig Drugs. 2003 4(8):944-52). Agents in development are numerous and include oxazolidinones (Diekema D J, Jones R N. “Oxazolidinone antibiotics” Lancet. 2001 8:358(9297):1975-82; Marchese A, Schito G C. “The oxazolidinones as a new family of antimicrobial agent” Clin Microbiol Infect. 20017 Suppl 4:66-74; Moellering R C. “Linezolid: the first oxazolidinone antimicrobial” Ann Intern Med. 2003 138(2): 135-42); tetracycline derivatives (e.g. GAR-93 6) (Zhanel G G, Homenuik K, Nichol K, Noreddin A, Vercaigne L, Embil J, Gin A, Karlowsky J A, Hoban D J. “The glycylcyclines: a comparative review with the tetracyclines” Drugs. 2004 64(1):63-88); cephalosporins (e.g., BAL9141 and RWJ-54428) (Glinka T W. “Novel cephalosporins for the treatment of MRSA infections” Curr Opin Investig Drugs. 2002 3(2):206-17); glycopeptides (e.g. oritavancin, LY-333328, dalbavancin) (Allen N E, Nicas T I. “Mechanism of action of oritavancin and related glycopeptide antibiotics” FEMS Microbiol Rev. 2003 26(5):51 1-32); streptogramins (e.g., Quinupristin/dalfopristin) (Blondeau J M, Sanche S E. “Quinupristin/dalfopristin” Expert Opin Pharmacother. 2002 3(9):1341-64); topoisomerase inhibitors (e.g., fluoroquinolones) (Harding I, Simpson I. “Fluoroquinolones: is there a different mechanism of action and resistance against Streptococcus pneumoniae?” Chemother. 2000 12 Suppl 4:7-15) and many others.

Recently, linezolid (Zyvox™, Pharmacia) and quinupristin-dalfopristin (Syncercid™; Aventis) were introduced into the clinical setting as alternatives for Gram-positive infections. The usefulness of these new agents, however, is tempered by their cost, toxicity, and concerns about further development of resistance (Rehm S J. “Two new treatment options for infections due to drug-resistant gram-positive cocci” Cleve Clin J Med. 2002 69(5):397-401). It has become clear that antibiotic therapy alone is insufficient to deal with the rise in bacterial disease (de Roux A, Lode H. “Recent developments in antibiotic treatment” Infect Dis Clin North Am. 2003 17(4):739-5 1). The need for novel therapeutics is readily apparent.

Gram-Negative Bacteria

Gram-negative organisms have thinner cell walls which differ in composition from their Gram-positive counterparts. As a result, Gram-negative bacteria lose the crystal violet stain (and take the color of the red counterstain) in Gram's method of staining. Clinically relevant Gram-negative bacteria including Salmonella, Shigella, Escherichia, Klebsiella, Enterobacter, Serratia, Proteus, Morganella, Providencia, Yersinia, Neisseria, Moraxella (Branhamella), the Acinetobacter and other less common genera.

Gram-negative bacilli account for many nosocomial pneumonias, including fatal ones. Gram-negative bacillary pneumonias typically occur in infants, the elderly, or other immunocompromised hosts. The most important pathogen is Klebsiella pneumoniae, which causes Friedlander's pneumonia. Other Gram-negative pathogens associated with pneumonia include Pseudomonas aeruginosa, Escherichia coli, Enterobacter sp., Proteus sp., Serratia marcescens, and Acinetobacter sp.

Escherichia coli, which normally inhibits the gastrointestinal tract, is another important cause of human infection. E. coli with colonizing, enterotoxic, cytotoxic, or invasive virulence traits can are associated with diarrhea, including hemolytic-uremic syndrome. Disruption of anatomical barrier may cause the organism to invade the bloodstream and spread to adjacent tissues. E. coli frequently causes urinary tract infections and bacteremia, and can also causes sepsis and other diseases in neonates, particularly premature neonates. The Klebsiella, Enterobacter, and Serratia genera cause infections in the same sites as does E. coli, and they are also an important cause of bacteremia.

The Proteeae are Gram-negative organisms including at least three genera: Proteus (P. mirabilis, P. vulgaris, and P. myxofaciens), Morganella (M morganii), and Providencia (P. rettgeri, P. alcalifaciens, and P. stuartii). P. mirabilis causes most human infections. These organisms are often found in superficial wounds, draining ears, and sputum, frequently when patients have undergone antibiotic therapy. They can cause infections of the ears, mastoid sinuses, peritoneal cavities, and urinary tract, as well as bacteremia.

Among the members of the Salmonella genus, S. enteritidis is responsible for the vast majority (i.e., 85%) of all Salmonella infections in the United States. S. enteritidis typically causes gastroenteritis. Many serotypes of S. enteritidis (of which there are more than 2000) are referred to informally (and incorrectly) as species, including S. typhimurium, S. heidelberg, S. newport, S. infantis, S. agona, S. Montevideo, and S. saint-paul. Less common is infection with S. typhi, which produces typhoid fever. Associated with unhygienic conditions in communal food or water supplies, S. typhi can cause ulceration, hemorrhage, and intestinal perforation in severe cases. Other diseases caused by Gram-negative bacteria include cholera and plague, caused by infection with Vibrio cholerae, and Yersinia pestis, respectively.

Similar to Gram-positive bacterial infections, Gram-negative organisms exhibiting antibiotic resistance are a growing problem. First observed in Europe in the early 1980's, Enterobacteriaceae with resistance to a wide range of beta-lactams (e.g., penicillins, cephalosporins and carbapenems) have since spread worldwide.

Drug development directed toward resistant Gram-negative bacteria strains is limited. There are no new beta-lactams, monobactams, carbapenems, aminoglycosides, or fluoroquinolones which are specifically active against gram-negative bacilli or being developed for use against resistant gram-negatives (Bishai W. “Drug Discovery at IDSA 2000” A Report from the 38th Annual Meeting of the Infectious Diseases Society; Gilbert D N. The current and future antimicrobial arsenal for the treatment of infections caused by Gram-negatives. 38th Annual Meeting of the Infectious Diseases Society of America, Sep. 7-10, 2000, New Orleans, L A. [Abstract S81]). Peptides represent one narrow area of new drug development, including Bactercidal Permeability Inducing peptide (BPI), MSI-78 and nisin. Similar the Gram-positive infections, the need for novel therapeutics for the treatment of infections caused by Gram-negative bacteria is evident.

Passive Immunity

The principle of passive immunity provides that neutralizing antibodies from a different organism may be used to prevent or treat disease in another organism of the same or different species. Passive immunization has a long history in the treatment of disease. The earliest form of passive immunity involved the use of serum therapy in the late 1800's. Behring and Kitasato were the first to use passive immunization in the treatment of diphtheria in 1890. In the 1920's and 30's, sera produced in animals (e.g., horse, rabbit, sheep) was used to treat patients with measles and scarlet fever. At that time, serum was not known to contain antibodies; rather it was only observed that serum produced a beneficial therapeutic effect.

Blood is now understood as a liquid tissue containing cells, proteins, salts and various amounts of organic substances. The term plasma refers to that portion of the blood that remains after the cellular elements (i.e., red blood cells, white blood cells) have been removed, typically by centrifugation. Serum, in contrast, refers to the portion of the blood that remains after both the cellular elements and the clotting proteins are removed. Clotting will occur naturally when blood is added to a test tube, but can also be stimulated by clotting activators such as calcium. Serum contains water (98%), protein (6-8%), salts (0.8%), lipids (0.6%) and glucose (0.1%). Other molecules, including metabolites, hormones and enzymes are also present. Serum should be therefore be distinguished from plasma, although the two terms are sometimes used interchangeably incorrectly. Those skilled in the art of blood collection are familiar with the differences between the two, which determine how blood samples are collected. To prevent conversion to serum, plasma is typically collected in tubes coated with an anticoagulant, or anticoagulant is added to the blood shortly after collection. Common additives used to prevent coagulation include heparin, EDTA, citrate or oxalate. Serum is collected in uncoated tubes and permitted to clot, or in tubes with clotting activators to speed the process.

Serum contains a complex mixture of proteins that contains various proteins ranging in concentration over at least 9 orders of magnitude (Adkins, J N et al., Mol Cell Proteomics 2002 1(12):947-55). The most abundant serum proteins are albumins, which account for between 60-80% of total serum protein. Albumin is a small protein produced by the liver and responsible for transport of small molecules, such as calcium, around the body. Albumin also helps keep blood fluids from leaking out into tissue. Globulins are a second form of protein found in large quantities in serum. Larger than albumin, the globulins can be classified according to three major types: alpha, beta and gamma. Alpha and beta globulins mainly carry various lipids, lipid-soluble hormones and vitamins, and other lipid-like substances in the plasma. The alpha-1 fraction includes alpha-1 anti-trypsin and thyroxine binding globulin. The alpha-2 fraction contains haptoglobin, ceruloplasmin, HDL, and alpha-2 macroglobulin. The beta fraction includes transferrin. The gamma globulins consist primarily of the immunoglobulin (i.e., Gia, IBM, IgG). Protein levels may vary somewhat based on, for example, disease or nutritional state.

The early use of serum therapy declined in popularity with the discovery of antibodies, and the development of strategies such as pooling and monoclonal antibody technology. Numerous extraction and purification strategies have been developed to isolate serum proteins with various degrees of specificity. These strategies exploit the differences among proteins with respect to such characteristics as size, charge, and binding affinity, among others. Representative technologies include chromatography (i.e., gel filtration, affinity and ion exchange), precipitation and gel electrophoresis. Robert K. Scopes, Protein Purification: Principles and Practice, 3rd edition, Springer Verlag (1994). These techniques can be used alone or in combination. For example, ionic precipitation (i.e., using ammonium sulfate) is most often used early in a purification to permit some subset of proteins to be fractionated from the whole based on common solubility parameters, and is often followed by chromatography. Ionic precipitation can be used to obtain a crude extract of proteins that can then be further purified.

Intravenous immune globulin, (i.e., IGIV or IVIG) has been used to treat bacterial infections (Cometta A, Baumgartner J D, Glauser M P. “Polyclonal intravenous immune globulin for prevention and treatment of infections in critically ill patients” Clin Exp Immunol 1994 97: S1: 69-72). Immune globulins represent a fraction of the blood (i.e., IgG, IgA, IgM and IgE) and are prepared from the pooled plasma of large number of donors. IVIGs tend to have broad representations of antibodies, including for example, antibodies for ubiquitous pathogens. IVIG therapy depends upon the presence of high and consistent tires of antibodies to the pathogen of interest. Antibody concentrations, however, vary from lot to lot (Siber et al., “Use of immune globulins in the prevention and treatment of infections” Current Clinical Topics in Infectious Disease, Remington J S, Swartz M M, etds. Blackwell Scientific, Boston, 12:208-257 (1992).

WO 01/60156 (Davis) teaches methods and compositions for treating viral and bacterial infections using neutralizing antibodies produced in goats. The goats are immunized with a virus (e.g., HIV) or bacteria (e.g., Stapylococcus, E. coli, Streptococus). The blood of the immunized animal is then collected, and processed by standard extraction and purification methods (e.g., ammonium sulfate precipitation followed by dialysis or gel filtration) to produce an immunomodulatory composition enriched for heterologous neutralizing antibodies Other filings by Davis include WO 97/02839, WO 02/07760 and US 2002/006022. All teach processing of immunized animal sera to obtain immunoglobulin-enriched serum extracts suitable for in vivo use.

WO 03/004049 (Dalgleish) teaches that therapeutic activity of goat serum processed in the manner of Davis et al. (i.e., a serum extract) is dependent upon anti-HLA and/or anti-FAS antibodies. Dalgleish suggests that the anti-inflammatory effect of these antibodies prevents over-stimulation of the immune system by viral epitopes (gp 120), which resemble normal human HLA. The anti-HLA and/or anti-FAS antibody-enriched compositions of Dalgleish are said to be useful in treating a wide variety of diseases with inappropriately high HLA levels. Chronic infections such as viral, bacterial and tropical cancers are specified. See also WO 03/064472 (Daigleish).

U.S. Pat. No. 5,219,578 (Ansley) teaches a method of stimulating the immune system of mammals to ward off infectious disease (i.e., equine lower respiratory disease or ELRD in horses caused by a variety of opportunistic organisms; ovine footrot in sheeps and lambs caused by various serotypes of B. nodosus; bovine respiratory disease) using an IgG fraction of goat sera free from foreign or artificially induced antigens. Ansley teaches that the non-immunized goat sera induce non-specific activation of the immune system in the treated animal.

Given the epidemic nature of bacterial infections, and the serious consequences for the infected patient, there exists a need for new strategies to treat and prevent bacterial infection.

Therefore, it is an object of the present invention to provide a composition for the treatment and prevention of bacterial infection.

It is another object of the present invention to provide a method and use for treating and preventing bacterial infection.

It is another object of the present invention to provide a composition and method to treat patients infected bacteria that consists of a simple, cost-effective regimen of therapy, with minimal side effects and broad efficacy.

It is another object of the present invention to provide a method for the manufacture of a composition or medicament for the treatment and prevention of bacterial infections.

### SUMMARY OF THE INVENTION

A composition, method and use for the treatment and prevention of bacterial infections and other related conditions is disclosed. The composition, method and use provide a plasma or serum fraction derived from a mammal exposed to an inoculant, which fraction has been depleted of one or more high molecular weight proteins or biological agents present in the unprocessed plasma or serum. The composition, method and use of the present invention provide a simple, cost-effective regime for treatment of bacterial infections, including both Gram-positive and Gram-negative bacterial infections and related conditions, either alone or in combination with conventional treatments such as antibiotics.

The composition of the present invention, in one embodiment an immunoglobulin-depleted fraction of plasma or serum derived from a mammal exposed to an inoculant (e.g., a bacteria-bearing inoculant), differs from the prior art reviewed above which utilizes immunoglobulin concentrates or purified antibodies for the treatment of bacterial infections. Without being bound by any particular theory, it is believed that one or more biological agents present in the plasma or serum fraction depleted of one or more high molecular weight proteins or biological agents may generate a beneficial plethoric effect in vivo.

Accordingly, one aspect of the invention is a composition useful in the treatment or prevention of bacterial infections, which is a plasma or serum fraction derived from a mammal exposed to an inoculant, which fraction has been depleted of one or more high molecular weight proteins or biological agents present in the unprocessed plasma or serum.

The inoculant used to generate the plasma or serum fraction of the present invention may vary. In one embodiment, the inoculant is a bacteria-bearing inoculant. Bacteria-bearing inoculants include, without limitation, the blood, plasma or serum of a person infected with bacteria, a bacterial lysate, tissue from a person infected with bacteria, lysates of cysts or other inclusion bodies containing bacteria, a purified bacterial preparation grown in vitro, or a suspension of bacteria in saline, plasma or another biological fluid.

In one embodiment, the bacteria-bearing inoculant is a Gram-positive bacteria-bearing inoculant. Non-limiting examples of Gram-positive bacteria-bearing inoculants include Staphylococcus-bearing inoculants, Streptococcus-bearing inoculants and Enterococcus-bearing inoculants.

In a particular embodiment, the Staphylococcus-bearing inoculant is a Staphylococcus aureus-bearing inoculant. In a preferred embodiment, the inoculant is a methicillin-resistant Staphylococcus aureus-bearing inoculant.

In another particular embodiment, the Streptococcus-bearing inoculant is a Streptococcus pneumonia-bearing inoculant. In a preferred embodiment, the inoculant is a penicillin resistant Streptococcus pneumonia-bearing inoculant.

In yet another particular embodiment, the Enterococcus-bearing inoculant is a vancomycin-resistant Enterococcus-bearing inoculant.

In another embodiment, the bacteria-bearing inoculant is a Gram-negative bacteria-bearing inoculant selected from the group comprising Salmonella, Shigella, Escherichia, Klebsiella, Enterobacter, Serratia, Proteus, Morganella, Providencia, Yersinia, Neisseria, Moraxella (Branhamella), and Acinetobacter-bearing inoculants.

In a further embodiment, the inoculant is a viral-bearing inoculate such as an HIV-bearing inoculant.

The high molecular weight protein(s) or biological agent(s) depleted from the plasma or serum fraction of the present invention may vary. Representative, non-limiting high molecular weight proteins include immunoglobulin, albumin, transferrin, haptoglobin and lipoproteins.

The term “depletion” is used to indicate a reduction in the amount of a compound(s) or molecule(s) (e.g., high molecular weight proteins) in a given sample after the sample is treated according to the method of the present invention.

In one embodiment, the plasma or serum fraction is depleted of approximately 1-10%, 10-20%, 20-30%, 30-40%, 40-50%, 50-60%, 60-70%, 70-80%, 80-90%, or 90-100% of the one or more high molecular weight proteins present in the unprocessed plasma or serum sample.

In a particular embodiment, the present invention is a composition useful in the treatment of prevention of a bacterial infection or related condition which is a plasma or serum fraction derived from a mammal exposed to an inoculant, which fraction has been depleted of immunoglobulin present in the unprocessed plasma or serum.

Another aspect of the invention is a composition useful in the treatment or prevention of bacterial infections, which is a plasma or serum fraction derived from a mammal exposed to an inoculant (e.g., a bacteria-bearing inoculant or a viral-bearing inoculant), which fraction has been depleted of two or more different high molecular weight proteins or biological agents present in the unprocessed plasma or serum.

In a particular embodiment of the present invention, the plasma or serum fraction has been depleted of immunoglobulin and albumin present in the unprocessed plasma or serum sample.

In another aspect, the present invention is a composition useful in the treatment or prevention of bacterial infections which is a plasma or serum fraction derived from a mammal exposed to an inoculant (e.g., a bacteria-bearing inoculant or a viral-bearing inoculant), which fraction has been depleted of proteins or biological agents with a molecular weight greater than approximately 50 kD.

In another particular embodiment of the present invention, the composition useful in the treatment or prevention of bacterial infections is a plasma or serum fraction derived from a mammal exposed to an inoculant (e.g., a bacteria-bearing inoculant or a viral-bearing inoculant), which fraction has been depleted of proteins or biological agents with a molecular weight greater than approximately 30 kD.

In another embodiment of the present invention, the composition useful in the treatment or prevention of bacterial infections is a plasma or serum fraction derived from a mammal exposed to an inoculant (e.g., a bacteria-bearing or HIV-bearing inoculant), which fraction has been depleted of proteins or biological agents with a molecular weight greater than approximately 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 or 65 kD present in the unprocessed plasma or serum.

Another aspect of the present invention is a method of treating or preventing a bacterial infection or related conditions by administering to a subject in need thereof a therapeutic amount of a plasma or serum fraction derived by exposing a mammal to an inoculant, which fraction is depleted of one or more high molecular weight proteins or biological agents present in the unprocessed plasma or serum, either alone or in combination or alternation with another anti-bacterial agent or agent that treats a related condition.

In a particular embodiment, the subject is a human.

In one embodiment, the bacterial infection is a Gram-positive bacterial infection selected from the group consisting of Staphylococcus infections, Streptococcus infection, Enterococcus infections and related conditions.

In another embodiment, the bacterial infection is a Gram-negative bacterial infection selected from the group consisting of Salmonella, Shigella, Escherichia, Klebsiella, Enterobacter, Serratia, Proteus, Morganella, Providencia, Yersinia, Neisseria, Moraxella (Branhamella) and Acinetobacter infections and related conditions.

The composition of the present invention can be administered by any effective means, including but not limited to, subcutaneous, parenteral, intravenous, intraarterial or oral administration. In a particular embodiment, the composition is administered by subcutaneous injection.

In one embodiment, the inoculant is a viral-bearing inoculant (e.g., an HIV-bearing inoculant) or a bacteria-bearing inoculant (e.g., Gram-positive or Gram-negative bacteria-bearing).

In a particular embodiment, the plasma or serum fraction is depleted of immunoglobulin present in the unprocessed plasma or serum.

In another particular embodiment, the plasma or serum fraction is depleted of immunoglobulin and albumin present in the unprocessed plasma or serum.

Another aspect of the present invention is a method of preparing the composition useful in the treatment or prevention of bacterial infections, involving (a) exposing a mammal to an inoculant; (b) allowing time for the mammal to respond to the inoculant and to produce one or more beneficial biologic agents in the blood; and (c) obtaining the plasma or serum; (d) processing the plasma or serum to isolate the anti-bacterial activity from one or more high molecular weight proteins or biological agents present in the unprocessed plasma or serum.

In one embodiment, the mammal is an ungulate. In a preferred embodiment, the mammal is a goat.

In a particular embodiment, the mammal is not susceptible to infection with the inoculant.

In another embodiment, the inoculant is a bacteria-bearing inoculant. The bacterial-bearing inoculant may be, for example, a Gram-positive bacterial-bearing inoculant or a Gram-negative bacterial bearing inoculant. In a further embodiment, the inoculant is a viral-bearing inoculant, for example, an HIV-bearing inoculant.

The process used to isolate the anti-bacterial activity from the high molecular weight proteins or biological agents present in the unprocessed plasma or serum may vary. The process may include, without limitation, fractionation methods such as fractional precipitation, dialysis and ultrafiltration, and/or chromatographic fractionation. In a particular embodiment, the process includes ammonium sulfate precipitation. In another embodiment, the process includes gel filtration chromatography, ion exchange chromatography or affinity chromatography. The process may involve a single fractionation step or multiple fractionation steps involving the same or different fractionation methods.

In a preferred embodiment, the plasma or serum is processed to isolate the anti-bacterial virus activity from immunoglobulin and albumin present in the initial plasma or serum sample by sequential ammonium sulfate precipitation and DEAE-column chromatography.

### BRIEF DESCRIPTION OF THE FIGURES

FIG. 1 is a graphical representation of the protein profile obtained as described in Example 13 from a DG-10 desalting column of Week 3 serum from an animal inoculated as described in Example 11. The column was equilibrated with buffer A, 3 ml of serum was loaded onto the column and 1 ml fractions were collected. The protein concentration of each fraction was determined and the results were plotted vs. the approximate elution volume. Fractions representing milliliters 4 through 9 were pooled for DEA-blue chromatography.

FIG. 2 shows Coomassie blue stain of partially purified protein serum fractions. Protein from serum and partially purified fractions (as described in Example 13) was subjected to electrophoresis on a 6 to 18% polyacrylamide Tris-SDS gel. Following electrophoresis, the gel was stained with Coomassie G-250 to visualize the proteins. BR and P are Bio Rad broad range pre-stained molecular weight markers, and Pierce TriChromRanger molecular weight markers, respectively. The molecular weight in kilodaltons of the BioRad markers are indicated on the left hand side of the figure. IgG is 2 μg of purified goat IgG obtained from the NIH AIDS Research and Reference Reagent Program.

FIG. 3 is an immunoblot of partially purified serum fractions with anti-goat IgG, as described in Example 13. Protein from serum and partially purified fractions was subjected to electrophoresis on a 6 to 18% polyacrylamide Tris-SDS gel. Following electrophoresis, the gel was stained with Coomassie G-250 to visualize the proteins. BR and P are Bio Rad broad range pre-stained molecular weight markers, and Pierce TriChromRanger molecular weight markers, respectively. The molecular weight in kilodaltons of the BioRad markers are indicated on the left hand side of the figure. IgG is 2 μg of purified goat IgG obtained from the NIH AIDS Research and Reference Reagent Program.

FIG. 4 is a graphical representation of the chromatographic profile for the DEAE-Blue 30 column fractionation of the 66% ammonium sulfate pellet detailed in Example 15, from an animal inoculated as described in Example 11. The blue trace monitors absorbance at 254 nm vs time and represents the protein elution profile. The red trace monitors eluate conductivity vs. time and represents the ionic concentration of the wash or elution buffer.

FIG. 5 shows SDS-PAGE from the partial fractionation detailed in Example 15, from an animal inoculated as described in Example 11. Five micrograms of total protein for each fraction was electrophoresed on a 8-16% polyacrylamide gel. The gel was stained with Bio Safe Commassie stain over night at room temperature and destained with water. The image was captured using an Alpha Inotech gel imager. The molecular weights of dye-labeled protein standards (BioRad) is indicated on the left of the figure.

### DETAILED DESCRIPTION OF THE INVENTION

The present invention includes compositions, methods and uses for treating and preventing bacterial infections and related conditions. In a particular embodiment, the compositions and methods of the present invention are useful for treating and preventing Gram-positive and Gram-negative bacterial infections in subjects in need thereof, such as human patients. The plasma or serum fraction can be administered to a patient exposed to or infected with the bacteria, preventing development of infection or producing a decrease in the symptoms of bacterial disease and without producing significant side effects.

As used herein, the term Gram-positive bacterial infection and Gram-negative bacterial infection refers to infection with Gram-positive or Gram-negative bacteria, as typically diagnosed in a clinical setting. Diseases and conditions caused by such infections include, without limitation, pneumonia, meningitis, sepsis, bacterial endocarditis, streptococcal exudative pharyngitis, cellulites, wound infection, and visceral abscesses, acute rheumatic fever, poststreptococcal glomerulonephritis, urinary tract infections, septicemia, bacteremia, osteomyelitis, appendicitis, otitis media, colon cancer, strep throat, scarlet fever, impetigo, sinusitis, peritonitis, arthritis, strep pneumonia, pneumococcal pneumonia, pharyngitis, tonsillitis, mastoiditis, joint and bone infections, erysipelas, chorioarnnionitis, endometritis, skin and soft tissue infection, conjunctivitis, enterocolitis, toxic shock syndrome, peritonitis, diarrhea, hepatobiliary, peritoneal, cutaneous, and pulmonary infections, ear infections, mastoid sinus infections, headache, constipation, anorexia, abdominal pain and tenderness, dysuria, nonproductive cough, epistaxis, splenomegaly, leucopenia, anemia, liver function abnormalities, proteinuri, acute cholecystitis and hepatitis, pneumonia, osteomyelitis, soft tissue abscesses, glomerulitis, gastroenteritis, epiglottitis, bacteremic Brazilian purpuric fever, chancroid, encephalitis, neuritis, orchitis, cholecystitis, hepatic suppuration, mediastiitis, lung abscess, cholera, hypovolemia, renal tubular necrosis, plague, meliodosis, bronchitis, endocarditis, cellulites, sexually transmitted diseases, urethritis, cervicitis, proctitis, salpingitis, epididymitis, skin lesions, bone lesions, among others.

As used herein, “treatment” is an approach for obtaining beneficial or desired results, including clinical results. For purposes of this invention, beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

A. Process for Producing the Plasma or Serum Fraction

The following are non-limiting embodiments of how to carry out the invention. Given the description of the invention provided throughout this text, one of ordinary skill can modify the steps below without deviating form the spirit or scope of the invention.

(i) Selection of Animal

The plasma or serum fraction may be prepared using an animal, such as a mammal, that produces an effective product according to the process described in detail herein. In one embodiment of the invention, the mammal produces an effective product post-inoculation.

In a particular embodiment of the invention, the mammal or animal is not subject to infection with the inoculant. For example, when HIV is used as the inoculant, the mammal or animal is not subject to infection with HIV. Or, for example, when bacteria (e.g., a Gram-positive or Gram-negative bacteria) are as the inoculant, the mammal or animal is not subject to infection with the bacteria.

Non-limiting examples of animals suitable for this purpose include cow, rabbit, cat, dog, mouse, goat, lamb, sheep, horse, deer, pig, mouse, chicken and the like (for example Bora goats). In a particular embodiment, the mammal is an ungulate or hoofed-mammal. Non-limiting examples of ungulates include goats, sheep, horses, and cows.

In a particularly preferred embodiment, the mammal is a goat.

(ii) Inoculation of the Animals

The plasma or serum fraction may be prepared by inoculating an animal by any suitable means. Any inoculant suitable for generating the plasma or serum fraction can be used in the present invention. In a particular embodiment of the invention, the inoculant is a viral inoculant. Non-limiting examples of viral inoculants include the blood, plasma or serum of an individual infected with a virus, a viral lysate, purified virus or naturally occurring or synthetic viral proteins or peptides (glycosylated or unglycosylated). The virus may also be one of the following:

In one embodiment of the present invention, the viral inoculant is a Herpesviridae bearing inoculant, a Retroviridae bearing inoculant, a Flaviviridae bearing inoculant, an Orthomyxoviridae bearing inoculant, a Paramyxoviridae bearing inoculant, a Togaviridae bearing inoculant, a Picornaviridae bearing inoculant, a Coronaviridae bearing inoculant, an Adenoviridae bearing inoculant, a Poxviridae bearing inoculant, a Hepadnaviridae bearing inoculant, a Reoviridae bearing inoculant, a Parvoviridae (including a Parvovirinae and/or Densovirinae bearing inoculant), a Rhabdoviridae bearing inoculant, a Bunyaviridae bearing inoculant, a Bromoviridae bearing inoculant, a Totiviridae bearing inoculant, a Tectiviridae bearing inoculant, a Plasmaviridae bearing inoculant, a Myoviridae bearing inoculant, a Siphoviridae bearing inoculant, a Podoviridae bearing inoculant, a Rudiviridae bearing inoculant, a bearing inoculant, a Corticoviridae bearing inoculant, a Lipothrixviridae bearing inoculant, a Plasmaviridae bearing inoculant, a Fuselloviridae bearing inoculant, a Phycodnaviridae bearing inoculant, an Iridoviridae bearing inoculant, a Polydnaviridae bearing inoculant, a Polyomaviridae bearing inoculant, a Papillomaviridae bearing inoculant, a bearing inoculant, an Ascoviridae bearing inoculant, a Baculoviridae bearing inoculant, a Nimaviridae bearing inoculant, an Asfarviridae bearing inoculant, an Inoviridae bearing inoculant, a Microviridae bearing inoculant, a Geminiviridae bearing inoculant, a Circoviridae bearing inoculant, a Nanoviridae bearing inoculant, a Pseudoviridae bearing inoculant, a Metaviridae bearing inoculant, a Caulimoviridae bearing inoculant, a Cystoviridae bearing inoculant, a Birnaviridae bearing inoculant, a Totiviridae bearing inoculant, a Chrysoviridae bearing inoculant, a Partitiviridae bearing inoculant, a Hypoviridae bearing inoculant, a Filoviridae bearing inoculant, a Bomaviridae bearing inoculant, an Arenaviridae bearing inoculant, a Leviviridae bearing inoculant, a Dicistroviridae bearing inoculant, a Sequiviridae bearing inoculant, a Comoviridae bearing inoculant, a Potyviridae bearing inoculant, a Caliciviridae bearing inoculant, an Astroviridae bearing inoculant, a Nodaviridae bearing inoculant, a Tetraviridae bearing inoculant, a Tombusviridae bearing inoculant, an Arteriviridae bearing inoculant, a Roniviridae bearing inoculant, a Bromoviridae bearing inoculant, a Closteroviridae bearing inoculant, a Barnaviridae bearing inoculant, a Luteoviridae bearing inoculant, a Namaviridae bearing inoculant, a Pospiviroidae bearing inoculant, an Avsunviroidae bearing inoculant, and/or a prion bearing inoculant.

In a sub-embodiment of the present invention, the viral inoculant is a Herpesviridae bearing inoculant. In a particular embodiment of the invention, the viral inoculant is an HSV-1 or HSV-2 bearing inoculant. In another particular embodiment of the present invention, the viral inoculant is a human herpesvirus 3 (varicella-zoster virus) bearing inoculant. In yet another particular embodiment of the present invention, the viral inoculant is a CMV-bearing inoculant. In a still another particular embodiment of the present invention, the viral inoculant is an EBV—bearing inoculant. In a still another particular embodiment of the present invention, the viral inoculant is an human herpesvirus 6-bearing inoculant. In a still another particular embodiment of the present invention, the viral inoculant is an human herpesvirus 7-bearing inoculant. In a still another particular embodiment of the present invention, the viral inoculant is an human herpesvirus 8 (Kaposi's sarcoma-associated herpesvirus)-bearing inoculant.

In a sub-embodiment of the present invention, the viral inoculant is a Retroviridae bearing inoculant. In a particular embodiment of the invention, the viral inoculant is an HIV-bearing inoculant, wherein the HIV includes the many clades, types and subtypes of HIV. In a particular embodiment of the invention, the viral inoculant is an HIV-1 bearing inoculant (Clade A, B, C, D, F, H, and/or 0) and/or HIV-2 (Clade A and/or B) bearing inoculant. In another particular embodiment of the invention, the viral inoculant is an Human T-lymphotropic virus 2 (HTLV-2) bearing inoculant.

In another sub-embodiment of the present invention, the viral inoculant is a Flaviviridae bearing inoculant. In a particular embodiment of the invention, the viral inoculant is a flavivirus bearing inoculant, wherein the flavivirus is, for example, a Dengue virus (Dengue virus, Dengue virus type 1, Dengue virus type 2, Dengue virus type 3, Dengue virus type 4), a Japanese encephalitis virus (Alfuy Virus, Japanese encephalitis virus, Kookaburra virus, Koutango virus, Kunjin virus, Murray Valley encephalitis virus, St. Louis encephalitis virus, Stratford virus, Usutu virus, West Nile Virus), a Modoc virus, a Rio Bravo virus (Apoi virus, Rio Brovo virus, Saboya virus), a Ntaya virus, a Tick-Borne encephalitis (tick born encephalitis virus), a Tyuleniy virus, an Uganda S virus and/or a Yellow Fever virus. In another particular embodiment of the invention, the viral inoculant is a hepacivirus bearing inoculant, wherein the hepacivirus is, for example, an hepatitis C virus (HCV) and/or its many clades, types and subtypes. In yet another particular embodiment of the invention, the viral inoculant is a pestivirus bearing inoculant, wherein the pestivirus is, for example, Bovine Viral Diarrhea Virus-2 (BVDV-2), Pestivirus type 1 (including BVDV), Pestivirus type 2 (including Hog Cholera Virus) and/or Pestivirus type 3 (including Border Disease Virus).

In yet another sub-embodiment of the present invention, the viral inoculant is a Orthomyxoviridae bearing inoculant. In a particular embodiment of the invention, the viral inoculant is an Influenzavirus A bearing inoculant. In another particular embodiment of the invention, the viral inoculant is an Influenzavirus B bearing inoculant. In yet another particular embodiment of the invention, the viral inoculant is an Influenzavirus C bearing inoculant. In yet another particular embodiment of the invention, the viral inoculant is an Influenzavirus D bearing inoculant.

In yet another sub-embodiment of the present invention, the viral inoculant is a Paramyxoviridae bearing inoculant. In a particular embodiment of the invention, the viral inoculant is a Paramyxovirnae bearing inoculant. In an even more particular embodiment of the invention, the viral inoculant is a paramyxovirus, wherein the paramyxovirus is, for example, a Sendai virus, such as human parainfluenza virus 1 and human parainfluenza virus 3. In an even more particular embodiment of the invention, the viral inoculant is a human parainfluenza virus 1 bearing inoculant. In another even more particular embodiment of the invention, the viral inoculant is a human parainfluenza virus 3 bearing inoculant. In another particular embodiment of the invention, the viral inoculant is a rubulavirus bearing inoculant. In an even more particular embodiment of the invention, the viral inoculant is a human parainfluenza virus 2 bearing inoculant. In another even more particular embodiment of the invention, the viral inoculant is a human parainfluenza virus 4 bearing inoculant. In an even more particular embodiment of the invention, the viral inoculant is a mumps virus bearing inoculant. In another particular embodiment of the invention, the viral inoculant is a morbilli˜rurs bearing inoculant. In more particular embodiment of the invention, the viral inoculant is a measles virus bearing inoculant. In another particular embodiment of the invention, the viral inoculant is a Pneumovirnae bearing inoculant.

In a more particular embodiment of the invention, the viral inoculant is a respiratory syncytial virus (RSV) bearing inoculant.

In yet another sub-embodiment of the present invention, the viral inoculant is a Coronaviridae bearing inoculant. In a particular embodiment of the invention, the viral inoculant is a human respiratory coronavirus (HCV-229E) bearing inoculant. In another particular embodiment of the invention, the viral inoculant is a human respiratory coronavirus (HCV-0C43) bearing inoculant. In yet another particular embodiment of the invention, the viral inoculant is a torovirus bearing inoculant, such as a human torovirus bearing inoculant.

In yet another sub-embodiment of the present invention, the viral inoculant is a Togaviridae bearing inoculant. In a particular embodiment of the invention, the viral inoculant is a aiphavirus bearing inoculant. In another particular embodiment of the invention, the viral inoculant is a rubivirus bearing inoculant. In an even more particular embodiment of the invention, the viral inoculant is a Rubella virus bearing inoculant. In another even more particular embodiment of the invention, the viral inoculant is a Sindbis virus bearing inoculant. In another even more particular embodiment of the invention, the viral inoculant is EastemlWestern encephalitis virus bearing inoculant.

In yet another sub-embodiment of the present invention, the viral inoculant is a Picornaviridae bearing inoculant. In a particular embodiment of the invention, the viral inoculant is a human rhinovirus bearing inoculant, wherein the human rhinoviruses can be any one of the at least 105 serotypes (a classification scheme based on the variation of surface epitopes), which represent the most common etiological agent for the common cold. In a particular embodiment of the invention, the viral inoculant is an enterovirus. In an even more particular embodiment of the invention, the viral inoculant is a Human polioviruses 1, 2, and 3 (A23-echovirus; echo=Enteric Cytopathic Human Orphan viruses) (3 serotypes), Human coxsackieviruses A1-22, 24 (23 serotypes), Human coxsackieviruses B1-6 (swine vesicular disease virus is very similar to coxsackie B5 virus) (6 serotypes), Human echoviruses 1-7, 9, 11-27, 29-34 (30 serotypes; these viruses show a seasonal, epidemic pattern of infection primarily associated with meningitis, paralysis (usually less severe than acute poliomyelitis), and myocarditis), Human enteroviruses 68-71 (4 serotypes), and/or Vilyuisk virus (1 serotype) bearing inoculant. In a particular embodiment of the invention, the viral inoculant is cardiovirus bearing inoculant, wherein the cardiovirus can be, for example, an encephalomyocarditis (EMC) virus (a mouse virus that can infect humans, elephants, and squirrels; includes mengovirus, Maus-Elberfield virus, and the Columbia virus) and Theiler's murine encephalocyelitis (TME) virus (TO, GDVII). In another particular embodiment of the invention, the viral inoculant is hepatovirus bearing inoculant. In yet another particular embodiment of the invention, the viral inoculant is human hepatitis virus A bearing inoculant. In yet another particular embodiment of the invention, the viral inoculant is a severe acute respiratory syndrome (SARS) Co-V bearing inoculant.

In yet another sub-embodiment of the present invention, the viral inoculant is a Hepadnaviridae bearing inoculant. In a particular embodiment of the invention, the viral inoculant is a human hepatitis B virus (HBV) bearing inoculant.

In yet another sub-embodiment of the present invention, the viral inoculant is a Adenoviridae bearing inoculant. In a particular embodiment of the invention, the viral inoculant is a human adenovirus A, B, C, D, B, and/or F bearing inoculant.

In yet another sub-embodiment of the present invention, the viral inoculant is a Arenaviridae bearing inoculant. In a particular embodiment of the invention, the viral inoculant is a human hepatitis D virus (HDV) bearing inoculant.

In yet another sub-embodiment of the present invention, the viral inoculant is a Caliciviridae bearing inoculant. In a particular embodiment of the invention, the viral inoculant is a human hepatitis E-virus bearing inoculant.

In yet another sub-embodiment of the present invention, the inoculant is a prion bearing inoculant. In a particular embodiment of the invention, the inoculant is a prion bearing inoculant, wherein the prion is the causative agent of a spongiform encephalopathy such as Scrapie, Bovine spongiform encephalopathy (BSE), mad cow disease, Kuru, Creutzfeldt-Jakob disease (CJD), Gerstmann-Sträussler-Scheinker syndrome (GSS), and/or Fatal familial insomnia (FF1).

In another embodiment of the present invention, the inoculant is a bacteria-bearing inoculant. Non-limiting examples of inoculants include the blood, plasma or serum of a person infected with bacteria, a bacterial lysate, tissue from a person infected with a bacteria, lysates of cysts or other inclusion bodies containing bacteria, a purified bacterial preparation grown in vitro, or a suspension of bacteria in saline, plasma, or another biological fluid.

In a preferred embodiment of the present invention, the inoculant is a Gram-positive bacteria-bearing inoculant. The Gram-positive bacteria-bearing inoculant may be based on any Gram-positive bacteria. Non-limiting examples of Gram-positive bacteria that may be used to prepare the Gram-positive bacterial inoculant include: members of the Staphylococcus genus (e.g., Staphylococcus aureus, Staphylococcus epidermidis, S. haemolyticus, S. hominis, S. exotoxin and S. saprophyticus); the Streptococcus genus (e.g., Streptococcus pyogenes, Streptococcus pneumoniae, Streptococcus agalactia, Streptococcus mutans); the Enterococcus genus (e.g., E. faecium, E. faecalis, E. avium, E. casseliflavus, E. durans, E. gallinarum, E. dispar, E. hirae, E. flavescens, E. mundtii, E. solitarius, E. raffinosus); Peptostreptococcus sp. (e.g. Peptostreptococcus magnus, Peptostreptococcus asaccharolyticus, Peptostreptococcus anaerobius, Peptostreptococcus prevotii, and Peptostreptococcus micros.); Veillonella; catalase-negative gram-positive cocci including viridans streptococcal species such as S. mutans and S. sobrinus, S. salivarius and S. vestibularis, S. bovis, S. pneumoniae, S. sanguis and S. gordonii, S. mitis and S. oralis, S. anginosus, S. constellatus, and S. intermedius, S. milleri, S. MG-intermedius, S. anginosus-constellatus; Abiotrophia and Granulicatella; the Gemella genus (e.g., Gemella haemolysans, Gemella morbillorum, Gemella bergeriae, Gemella sanguinis); Rothia mucilaginosa; Aerococcus (e.g., Aerococcus viridans, A. urinae); Lactococcus (e.g., L. lactis, L. s garviae); Helcococcus (e.g., Helcococcus kunzii); the genus Globicatella (e.g., Globicatella sanguis); Facklamia; Ignavigranum; Dolosicoccus; Dolosigranulum (e.g., Dolosigranulum pigrum); Alloiococcus (e.g., A. otitidis); Vagococcus (e.g., V. fluvialis and V. salmoninarum); Leuconostoc (e.g., L. citreum, L. lactis, L. mesenteroides, L. pseudomesenteroides, L. argentinum and L. paramesenteroides); Pediococcus (e.g., P. acidilactici and P. pentosaceus), Tetragenococcus (e.g., Tetragenococcus halophilus), Lactobacillus sp., Clostridium sp. (Clostridium botulinum, Clostridium botulinum, Clostridium perfringens, Clostridium tetani); Actinomyces sp.(e.g., A. Israeli), Bifidobacterium (e.g., B. dentium), Nocardia sp, Listeria monocytogenes, Corynebacterium diptheriae, Propionibacterium acnes; Bacillus anthracis, and Erysipelothrix rhusiopathiae, as well as other clinically-relevant Gram-positive cocci well known in the art.

In a preferred embodiment, the Gram-positive bacteria bearing inoculant is a Staphylococcus aureus-bearing inoculant. In a particularly preferred embodiment, the Gram-positive bacteria bearing inoculant is a methicillin-resistant Staphylococcus aureus-bearing inoculant (i.e., the inoculant contains a methicillin-resistant Staphylococcus aureus immunogen).

In another preferred embodiment, the Gram-positive bacteria bearing inoculant is a Streptococcus pneumonia-bearing inoculant. In a particularly preferred embodiment, the Gram-positive bacteria bearing inoculant is a penicillin-resistant Streptococcus pneumonia-bearing inoculant (i.e., the inoculant contains a penicillin-resistant Streptococcus pneumonia immunogen).

In another embodiment of the invention, the inoculant is a Gram-negative bacteria-bearing inoculant. The Gram-negative bacteria bearing inoculant may be based on any Gram-negative bacteria, including without limitation: Klebsiella (e.g., K pneumoniae); Citrobacter; Serratia; Enterobacter; Proteus (P. mirabilis, P. vulgaris, and P. myxofaciens); Morganella (e.g., M morganii); Providencia (P. rettgeri, P. alcalifaciens, and P. stuartii); Salmonella sp. (e.g., S. typhi, S. paratyphi A, B S. schottmuelleri, S. hirschfeldii, S. enteritidis); Salmonella sp. (e.g., S. enteritidis S. typhimurium, S. heidelberg, S. newport, S. infantis, S. agona, S. montevideo, and S. saint-paul); the Shigella genus (e.g., S. fiexneri, S. sonnei, S. boydii, S. dysenteriae); the Haemophilus genus (e.g., H. influenzae); Brucella sp. (e.g., Brucella abortus, B. melitensis, B. suis, B. canis); Francisella tularensis; Vibrio sp. (e.g., V. cholerae, V. parahaemolyticus, V. mimicus, V. alginolyticus, V. hollisae, V. vulnificus); Yersinia sp. (e.g., Y. pestis, Y. enterocolitica); Burkholderia sp. (e.g., B. pseudomallei, B. cepacia); Campylobacter sp. (e.g., C. fetus, C. jejuni, C. coli); Helicobacter pylon; Serratia marcescens; Acinetobacter baumannii, Actinobacillus actinomycetemcomitans, Bordetella pertussis; Capnocytophaga; Cardiobacterium hominis; Eikenella corrodens; Kingella kingii; Legionella pneumophila; Pasteurella multisided; Acinetobacter; Xanthomonas maltophilia; Aeromonas; Plesiomonas shigelloides, Neisseria sp.(e.g., N. gonorrhoeae and N. meningitides), Moraxella (Branhamella) catarrhalis, Veillonella sp. (e.g., Veillonella parvula) and Acinetobacter sp.

In a particular embodiment, the inoculant contains two or more immunogens. These immunogens are different. According to one embodiment, one of these immunogens is a viral immunogen. In a preferred embodiment, the viral immunogen is an HIV immunogen. In a preferred embodiment, the inoculant comprises a viral immunogen and a bacterial immunogen. In a particularly preferred embodiment, the inoculant contains a HIV immunogen in combination with a Gram-positive bacteria-bearing immunogen.

In a preferred embodiment, the inoculant is plasma from a person infected with a virus or a bacteria. In one embodiment, the inoculant is plasma from a person infected with HIV or a person infected with a bacteria (e.g., a Gram-positive or Gram-negative bacteria).

According to one embodiment of the present invention, human blood is drawn from a virus or bacteria-infected patient using standard, sterile, phlebotic techniques. Preferably, the viral positive donors are between 18 and 65 years of age. Preferably, donors should appear healthy, not be under the influence of drugs or alcohol, and weigh in excess of 50 kg (110 lb). The following criteria of health can also be useful: body temperature less than 37.5° C.; pulse regular (50 to 100 beats per minute); blood pressure lower than 180 mm Hg systolic and 100 mm Hg diastolic; hemoglobin greater than 12.5 g/l and hematocrit greater than 38%. The blood is then processed to produce plasma according to techniques well known to those skilled in the art.

The plasma obtained from a virus-positive or bacterially-infected person can then be used to inoculate a mammal, such as a goat. The plasma can be injected one or more times. In addition, various adjuvants can be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and corynebacterium parvum. Such adjuvants are also well known in the art. Additional non-limiting examples of adjuvants suitable for use in the present invention are discussed further below.

The plasma and/or adjuvant can be injected in the mammal by one or more subcutaneous or intraperitoneal injections, though they can also be given intramuscularly, and/or intravenously.

The mammal can be given a sedative, for example Rompun, to facilitate handling of the mammal if necessary.

Preferably, at least 1 cc of human plasma can be administered to the mammal. For example, between 1-10 cc of the plasma can be administered to the animal subcutaneously. Alternatively, at least 1, 2, 5, 7, 10, 15, 20, 25, 30, 40 or 50 cc of plasma is administered subcutaneously, intraperitoneally intramuscularly, and/or intravenously.

In a particular embodiment, the animal is inoculated and then re-inoculated after a period of time ranging from 1, 2, 3, 4, 5, 6, 7, 8, 9, 10, 11, 12 or more weeks. The second inoculation is also known as a booster.

In one embodiment of the invention, an inoculant is not used. Rather, the serum or plasma fraction is prepared by obtaining plasma or serum from an animal, without prior inoculation.

(iii) Monitoring of Animal

The animal should preferably be monitored to indicate the patient sample with which it was injected and the date of injection and the animal should be monitored over a time period, beginning at about 1 week. Blood samples can be obtained from the animal during this time to measure the generated immune response. For example, the plasma from the blood sample can be measured for the ability to inhibit Gram-positive or Gram-negative bacterial growth in vitro using, for example, bacterial growth inhibition assays that measure the growth rate of bacteria in suspension culture by measuring optical density and by quantifying bacterial number by colony counting assay on solid agar plates. These assays are well known in the art. The mammal can be a goat. The time period of incubation can range from 1-8, 9, 10, 11, 12, 13, 14, 15, 16, 17, 18, 19, 20, 21, 22, 23, 24, 25, 26, 27 or 28 weeks. It is known that three weeks is a standard period of incubation for generating a sufficient immune response in goats.

One can assess the material using the procedures of various techniques are known in the art that include, but are not limited to: ELISA and Western blot. Other types of immunoassays known in the art can be used, including but not limited to, competitive and non-competitive assay systems using techniques such as radioimmunoassays, ELISA (enzyme linked immunabsorbant assay), “sandwich” immunoassays, immunoradiometric assays, gel diffusion precipitation reactions, immunodiffijsjon assays, in situ inimunoassays.(using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc.

An ELISA is a technique that uses antigens to coat the well of plates. ELISAs involve coating the well of a multiwell, such as a 96-well, microtiter plate with the antigen, washing away antigen that did not bind the wells, adding the blood or blood product from the mammal that has been inoculated conjugated to a detectable compound such as an enzymatic substrate (e.g., horseradish peroxidase or alkaline phosphatase) to the wells and incubating for a period of time, washing away unbound or non specifically bound materials, and detecting the presence of the specifically bound blood or blood product to the antigen coating the well. Alternately, in ELISAs the blood or blood product from the mammal that has been inoculated does not have to be conjugated to a detectable compound; instead, a second antibody (which recognizes the blood or component of the blood) can be conjugated to a detectable compound and added to the well. One of skill in the art would be knowledgeable as to the parameters that can be modified to increase the signal detected as well as other variations of ELISAs known in the art. For further discussion regarding ELISAs see, e.g., Ausubel et al, eds, 1994,10 Current Protocols in Molecular Biology, Vol. 1, John Wiley & Sons,—Inc., New York Bollag, D. M., Rozycki, M. D., and Edelstein, S. J. (1996). Protein Methods, Second Edition. New York: Wiley-Liss, 195-227.

Another useful technique is a western blot analysis. Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA. or non-fat milk), washing the membrane in washing buffer (e.g., PBS-Tween 20), applying the proposed binding protein (diluted in blocking buffer) to the membrane, washing the membrane in washing buffer, applying a secondary antibody (which recognizes the viral protein that you are assaying for) conjugated to an enzymatic substrate (e.g., horseradish peroxidase or alkaline phosphatase) or radioactive molecule (e.g. ¹²⁵I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc, New York 30 ; Harlow, E. and Lane, D. (1988). Antibodies: A Laboratory Manual. Cold Spring Harbor, N.Y.: Cold Spring Harbor Laboratory Press, 471-510.

Other techniques for evaluating immune responses in mammals are known to one skilled in the art.

(iv) Removal of Blood From Inoculated Animal

To obtain the plasma from the mammal, blood has to be collected. Any means to do this which accomplishes the desired goal is suitable. It is preferable to obtain large quantities of blood from the mammal, for example 10-30 cc of blood from a rabbit or similar sized animal and higher quantities from larger animals. The blood should begin to flow immediately through the tubing to the syringe, vacutainer, or open tube/bottle. If a syringe is used, gently draw on the syringe to collect the blood, and once the syringe is full, change syringes by disconnecting from the infusion set or needle hub. (See, for example, McGuill, M. W. and Rowan, A. N., “Biological Effects of Blood Loss: Implications for Sampling Volumes and Techniques,” ILAR News, Vol. 31(4), Fall 1989, pp 5-20).

In a particular embodiment of the present invention, the blood is collected in a container that prevents coagulation in order to obtain plasma not serum. A variety of methods are known in the art for preventing coagulation of drawn blood, and include, without limitation, collecting the blood in tubes or other types of collecting means that have been treated with an anticoagulant. Anticoagulant coated test tubes of this type are widely available commercially. Suitable anticoagulants include, but are not limited to EDTA, heparin, citrate or oxalate. Tube inversions allow proper mixing of anticoagulant additives and blood. Alternatively, a syringe and the infusion set tubing used in harvesting the blood can be filled with anticoagulant to aid in the harvesting of the plasma. Alternatively, the blood can be collected into a vacutainer or bottle that has been treated with anticoagulant. Alternatively, anticoagulants can be added to the plasma component of the blood after the cellular elements have been removed, for example by centrifugation as described below.

It is has been observed that plasma may not remain anticoagulated over time (i.e., it may clot to produce serum) unless proper techniques are utilized. Techniques for preventing plasma instability are known to those skilled in the art.

In one embodiment of the present invention, the composition is a plasma or serum fraction. When serum is desired, as opposed to plasma, the blood should be collected in a way that permits coagulation. Blood will naturally coagulate in a collection tube as coagulation factors become activated upon contact with a negative surface (“contact activation”). The time required to clot plasma may vary, and may range from less than a minute to more than an hour. Alternatively, the clotting process can be accelerated by the addition of a clotting activator to the tube or other container used to collect blood. Non-limiting examples of suitable clotting activators include calcium or silica particles.

The animal can be sedated. For example, 0.5 cc Rompun can be used to sedate, for example, a goat. In another example, Torbugesic (butorphanol; 1 mg/kg) and acepromazine maleate (1 mg/kg) can be used to sedate, for example, a rabbit. After the animal is sedated, the blood can be collected. One way to remove blood from an animal is to cannulate an artery, for example the external jugular artery. The mammal can be a goat and for a goat, an at least 18 gauge needle can be used to extract at least 150 cc of blood, preferable between 200-400 cc of blood. In another example, a needle, at least 21 gauges, is connected to an infusion device, such as an E-Z infusion set, to a syringe, for example, at least a 10 or 20 cc syringe.

As the blood is collected, it is preferably stored in a cooled environment, for example on ice or in a refrigerator or freezer.

(v) Treatment of Conditioned Blood to Form Plasma or Serum

The following description describes one way in which the blood can be treated to form plasma or serum.

In one embodiment, plasma is separated from blood by centrifugation. Centrifugation speeds and times are known to those skilled in the art, and may depend, for example, upon the type of tube used for blood collection. The specific gravity ranges for red cells are sufficiently different to enable isolation by centrifugation. Plasma is then obtained from the appropriate fragment.

In one embodiment of the present invention, the plasma can be repeatedly centrifuged to minimize the number of residual cells in the plasma fraction.

In another embodiment, serum is separated from clotted blood by centrifugation. Certain types of tubes known to those in the art may facilitate the separation process. For example, tubes containing a gel substance such that when the tube is centrifuged the cells go below the gel while the serum remains above.

(v) Treatment of Plasma or Serum to Form a High Molecular Weight-Protein Depleted Fraction

The anti-bacterial activity of the plasma or serum is then isolated from one or more of the various high molecular weight proteins (e.g., immunoglobulin, albumin) or biological agents present in serum or plasma. Plasma contains a mixture of hundreds of different kinds of proteins. (For a review, see Turner, M. W., and Hulme, B. (1970) The Plasma Proteins: An Introduction, Pitman Medical & Scientific Publishing Co., Ltd., London). Serum differs from plasma in that the clotting proteins (i.e., fibronectin) have been removed. The protein content of serum is approximately 60-80 mg/ml. The majority of serum protein is represented by a few, very abundant high molecular weight (HMW) proteins. Common high molecular weight proteins include, for example, immunoglobulins, albumin, transferrin, haptoglobin and lipoproteins.

Immunoglobulins (antibodies) are globular glycoproteins found in body fluids such as serum or on B cells where they act as antigen receptors. Immunoglobulins represent 10-25% of all serum proteins. They range in molecular weight from approximately 150,000-970,000 daltons. The five major classes of immunoglobulins (IgA, IgG, IgM, IgD and IgE), are distinguished by differences in the C regions of H chains of the molecule. They differ in size, charge, amino acid composition and carbohydrate content.

IgG is the dominant immunoglobulin (70-75%) in extracellular fluids like serum and has a molecular weight of approximately 150,000 daltons. IgM is the largest immunoglobulin, and has a molecular weight of 900,000 daltons. It represents approximately 10% of the total immunoglobulin pool. IgA concentrates in body fluids such as tears, saliva, and the secretions of the respiratory and gastrointestinal tracts. IgD accounts for less than 1% of the plasma immunoglobulins, and is almost exclusively found inserted into the membrane of B cells. IgE is normally present in only trace amounts, but it is responsible for the symptoms of allergy.

Albumin is a highly-water soluble protein with a molecular weight of approximately 66,000 Da. (For a review, see Peters T., Jr. All about Albumin: Biochemistry, Genetics, and Medical Applications Academic Press, San Diego, 1996) It is the most abundant protein in human blood, representing more than 55% of total serum proteins. It plays a role in the osmotic pressure of the plasma, and also functions as a carrier for hormones, enzymes, fatty acids, and metal ions. The average concentration of albumin in human serum is 4.0-4.8 g/100 ml.

Transferrin is a metal-binding glycoprotein with a molecular weight of approximately 80,000 daltons. (For a review, see Huebers H A and Finch C A. Physiological Reviews (1987) 67: 520). The primary function of transferrin is the transport of iron in plasma. It is also known as siderophilin.

Haptoglobin is a 100,000 dalton glycoprotein. It removes free hemoglobin from the circulation of vertebrates which binds free hemoglobin, preventing loss in the urine. Other diverse properties of human haptoglobin have been observed (see, e.g., Oh S K et al. J. Leuko. Biol., (1990) 47: 142-148; Cid M C et al. J. Clin. Invest. (1993) 91: 977-985).

Lipoproteins are lipid-protein complexes which permit the transport of otherwise insoluble lipids through the blood stream. The major serum lipoproteins include chylomicrons, very low density lipoproteins (VLDL), low density lipoproteins (LDL), intermediate-density lipoproteins (DL), and high-density lipoproteins (HDL).

Low molecular weight proteins found in plasma and serum include cytokines, chemokines, peptide hormones, as well as proteolytic fragments of large proteins. Cytokines and growth factors are typically between 6 and 50 kD, and more commonly between 10 and 3 OkD. The molecular weight of various low molecular weight proteins commonly found in human serum is detailed in the commonly available BioSource catalog.

Proteins can be separated from plasma or serum by fractionation. Fractionation strategies can vary in specificity, from very general to highly specific for a particular activity of interest. Fraction methods can be used alone or in combination. The goal of fractionation is to obtain a fraction enriched for an activity of interest. In the present invention, the activity of interest is believed to reside in a fraction of plasma or serum depleted of one or more high molecular weight proteins or biological agents such as albumins and immunoglobulins (e.g., IgG and IgM). The activity of interest is believed to be a low molecular weight protein or biological agent.

In a particular embodiment of the present invention, the desired product of fractionation is a fraction enriched for a proteins and biological agents between approximately 6 and approximately 50 kD

In a particular embodiment of the present invention, the desired product of fractionation is a fraction enriched for proteins and biological agents between approximately 6 and approximately 30 kD.

In a particular embodiment of the present invention, the desired product of fractionation is a fraction enriched for proteins and biological agents between approximately 6 and approximately 20 kD.

In a particular embodiment of the present invention, the desired product of fractionation is a fraction enriched for proteins and biological agents approximately 6 and approximately 14 kD.

In a particular embodiment of the present invention, the desired product of fractionation is a fraction enriched for proteins and biological agents between approximately 6 and approximately 10 kD.

In a particular embodiment of the present invention, the desired product of fractionation is a fraction enriched for proteins and biological agents between approximately 30 and approximately 50 kD.

In a particular embodiment of the present invention, the desired product of fractionation is a fraction enriched for proteins and biological agents between approximately 30 and approximately 40 kD.

In a particular embodiment of the present invention, the desired product of fractionation is a fraction enriched for proteins and biological agents between approximately 15 and approximately 25 kD.

In a particular embodiment of the present invention, the desired product of fractionation is a fraction enriched for proteins and biological agents between approximately 20 and approximately 25 kD.

In one embodiment of the present invention, the desired product of fractionation is a fraction enriched for a protein(s) or biological agents(s) with a molecular weight of approximately 12.2 kD.

In another embodiment of the present invention, the desired product of fractionation is a fraction enriched for a protein(s) or biological agent(s) with a molecular weight of approximately 14.1 kD.

In a further embodiment of the present invention, the desired product of fractionation is a fraction enriched for a protein(s) or biological agent(s) with a molecular weight of approximately 28.6 kD In yet another embodiment of the present invention, the desired product of fractionation is a fraction enriched for a protein(s) or biological agent(s) with a molecular weight of approximately 29 kD.

In yet a further embodiment of the present invention, the desired product of fractionation is a fraction enriched for a protein(s) or biological agent(s) with a molecular weight of approximately 30.1 kD.

In a particular embodiment of the present invention, the desired product of fractionation is a fraction enriched for a protein(s) or biological agent(s) with a molecular weight of approximately 49.4 kD.

In another embodiment of the present invention, the desired product of fractionation is a fraction enriched for a protein(s) or biological agent(s) with a molecular weight of approximately 53 kD.

It may require multiple fractionation steps to isolate the anti-bacterial activity from one or more of the high-molecular weight proteins or biological agents. Specifically, the anti-bacterial activity may initially fractionate with the high molecular weight proteins or biological agents, which high molecular weight fraction must then be further processed to isolate the anti-bacterial activity from the high molecular weigh proteins. Alternatively, the anti-bacterial activity may fractionate from the high molecular weight proteins or biological agents in a single fractionation step. Or, some of the activity of interest may initially fractionate with the high molecular weight fraction, while some additional portion of the activity may remain in the low molecular weight fraction.

A wide variety of methods are available to fractionate plasma or serum to isolate proteins. Such methods can be broadly divided into those which divide the protein between two phases (e.g., a solid and liquid) and those which separate proteins by different rates of movement through a material, such as a chromatographic column or electrophoresis gel. Any method capable of achieving the desired result is considered suitable for use in the present invention. These methods can be used alone, or in combination. Fractionation can involve a single step, or multiple steps.

Fractional precipitation can be used to deplete the initial serum or plasma fraction of high molecular weight proteins and biological agents. Non-limiting examples of fractional precipitation methods include solvent, salt, isolectric, hydrophilic polymer and heat precipitation. All fractional precipitation methods rely on bringing protein out of solution by altering the medium to reduce its solubility. Once insoluble, the protein can be separated form the mixture by centrifugation or filtration. Organic solvent precipitation methods are suitable for use in the method of the present invention. Addition of the solvent results in a decrease in the dielectric constant of the medium, which produces a decrease in protein solubility. Solvents may include, for example, 2 methyl-2,4-pentane diol (MPD), Dimethyl Sulfoxide (DMSO) and ethanol. In a particular embodiment, cold alcohol fractionation or ethanol fractionation, also known as the Cohn-Oncley method, is used (Cohn E J et al. J Am Chem Soc 1946; 68: 459-75) This method involves the precipitation of proteins under varying conditions of ethanol and pH conditions.

A variety of cold ethanol fractionation methods are known in the art for isolating albumin and immunoglobulin from plasma (See e.g., Cohn E J et al. J Am Chem Soc (1946) 68: 459-75; Hink J H et al. Vox Sang (1957) 2: 174-86; Kistler P et al. Vox Sang (1962) 7: 414-24). The Cohn and Kitler methods are compared in More J E et al. In: Harris J R, ed. Blood Separation & Plasma Fractionation. New York: Wiley, 1991; 261-306). Coagulation factors are removed as cryoprecipitate on initial thawing of the plasma before cold ethanol fractionation. With either method, an initial low ethanol precipitation stage removes the fibrinogen from the source plasma. Immunoglobulins are precipitated by raising the ethanol concentration to 25% at pH 6.9 for the Cohn method or 19% at pH 5.85 for the Kistler and Nitschmann method, while albumin remains in solution. Albumin is then isolated from the majority of the other plasma contaminants (mainly alpha and beta globulins), which are precipitated by the further addition of ethanol to a final ethanol concentration of 40%. This is carried out in two stages in the Cohn process but as a single step in the Kistler and Nitschmann method. In a final step, the albumin is itself precipitated near its isoelectric point. In an alternate approach to solvent precipitation, unwanted proteins in a mixture might be specifically inactivated and denatured by an organic solvent, thus allowing the contaminating protein to be removed.

Proteins can also be separated from plasma or serum by salt precipitation. Protein solubility is a function of the physiochemical nature of the proteins, pH temperature and the concentration of the salt used. It also depends on whether the salt is Kosomtropic (stabilizes water structure) or Chaotropic (disrupts water structure). Many types of salts (e.g., ammonium sulfate) can be employed to effect protein separation and purification. Ammonium sulfate is common used because of it is highly soluble, relatively inexpensive and generally preserves protein function. Using the appropriate concentration range of the given salt, a protein of interest can be preferentially isolated from a protein mixture. According to this method, increasing amounts of ammonium sulfate are added to give a certain percentage saturated, followed by a period of time to permit proteins to precipitate, and a centrifugation step to collect the precipitate.

In one embodiment of the present invention, ammonium sulfate precipitation is used to isolate the anti-bacterial activity of the plasma or serum from one or more high-molecular weight proteins or biological agents present in the unprocessed plasma or serum. In one embodiment, a single ammonium sulfate precipitation step is sufficient to isolate the anti-bacterial activity of the plasma or serum from one or more high-molecular weight proteins or biological agents present in the unprocessed plasma or serum. In another embodiment, ammonium sulfate precipitation is used to in combination with one or more additional fractionation steps or methods, either the same or different, to isolate the anti-bacterial activity of the plasma or serum from one or more high-molecular weight proteins or biological agents present in the unprocessed plasma or serum. For example, sequential ammonium sulfate precipitation (“cuts”) may be used.

In a particular embodiment, ammonium sulfate precipitation is used to isolate the anti-bacterial activity of the plasma or serum from immunoglobulin present in the unprocessed plasma or serum. These immunoglobulin may include IgG, IgM or both IgG or IgM.

Hydrophilic polymers such as polyethylene glycol (PEG) can also be used to precipitate proteins according to the present invention. PEG varies in chain lengths from average mol wt 1000 to 40000. Those of higher molecular weight are frequently useful in concentration schemes, with the most common PEG6000.

Isoelectric precipitation can also be used to fractionate proteins in the present invention. In general, proteins are positively charged at a low pH and negatively charged at a high pH. A protein is the least soluble when the pH of the solution is at its isoelectric point, i.e., the pH at which a protein molecule has a net charge of zero.

Heat precipitation also permits isolation of proteins according to the present invention. This method is typically used to remove contaminating proteins from a protein-containing solution. The stability of different proteins at elevated temperature varies, and if the desired protein has a greater heat stability than contaminating proteins, incubation at elevated temperatures (e.g., 45-70° C.) for a period of (i.e., varying from a few minutes to a few hours) produces precipitation of the unwanted proteins.

Dialysis and ultrafiltration can also be used to provide low-resolution protein fractionation. In dialysis, the protein sample is enclosed in a bag consisting of a semipermeable membrane (made of cellulose) and exposed to a large volume of a desired buffer. The low-molecular weight compounds (buffering agents, salts) pass freely through the membrane pores whereas the protein is retained. In ultrafiltration methods, the pores are generally larger and allow smaller proteins to pass through. Ultrafiltration typically employs pressure to force the sample through. Membranes with various molecular weight cutoffs are commercially available (i.e., from less than 10 to more than 100 kDa). Centrifugal ultrafiltration has also been used to deplete serum of large, highly abundant proteins such as albumin. (Tirumalai R S et al. Molecular & Cellular Proteomics (2003) 2:1096-1103.

Chromatographic fractionation of plasma or serum can also be used to isolate proteins from serum or plasma according to the present invention. In general, chromatography refers to any of a number of methods in which solutes are fractionated by partitioning between a mobile or buffer and an immobile or matrix, phase. Column chromatography is one type, and involves passing the starting material through a column which is constantly being washed through with a suitable buffer. As the protein enters the column, it interacts with the matrix of the column which can take many forms. Types of chromatography suitable for use in the present invention include, without limitation, gel filtration, ion exchange, affinity,

(i) Gel filtration chromatography, also known as molecular exclusion or gel permeation chromatography, separates molecules on the basis of size. In this method, the stationary phase is a gel matrix with a well-defined range of pore sizes is used. Large proteins do not enter the pores of the chromatographic matrix, but pass through, into the interstitial space between the matrix beads; this space is also known as the void volume, V0. These large proteins migrate more rapidly than small molecules which diffuse into and back out of the resin and consequently are partly trapped and fall behind. Proteins of intermediate size will penetrate to varying degrees into the beads and thus are separated from each other on the basis of their size. Low-pressure gel beads are capable of separating molecules from a molecular weight of a few hundred to multimeric proteins weighing in the millions range. These gel filtration resins are made from a variety of materials, including dextran, agarose, and polyacrylamide and are available in various pore sizes. Commercial gels include Bio-Gel (Bio-Rad) and Sephadex/Sepharose (Amersham Pharmacja Biotech).

In one embodiment of the present invention, gel filtration is used to isolate the anti-bacterial activity of the plasma or serum from one or more high-molecular weight proteins or biological agents present in the unfractionated plasma or serum. High molecular weight proteins, including immunoglobulin and serum albumin, typically fractionate in the first eluted fractions to come off of the column. The lower molecular weight proteins, such as cytokines, typically come off of the column in the latter fractions.

In one embodiment, a single gel filtration step is used to isolate the anti-bacterial activity of the plasma or serum from one or more high-molecular weight proteins or biological agents present in the unprocessed plasma or serum. In another embodiment, a gel filtration step is used in combination with one or more additional fractionation steps or methods, either the same or different, to isolate the anti-bacterial activity of the plasma or serum from one or more high-molecular weight proteins present in the unprocessed plasma or serum.

(ii) Ion exchange chromatography involves the use of a stationary phase matrix with covalently linked anions or cations. Solute ions of the opposite charge in the mobile liquid phase are attracted to the resin by electrostatic forces. Under specific starting conditions of buffer, pH, and ionic strength, the net charge on the protein of interest can be manipulated to interact with the matrix. These conditions are well known to those in the art. IEC media are available in differing charges, pore sizes, and support strengths (i.e., low-pressure to high-pressure tolerant). Commercial sources of JEC media include Amersham-Pharmacia, Bio-Rad, Dionex, Hewlett Packard, Merck, Perseptive Biosystems, and TosoHaas, among others.

In one embodiment of the present invention, the anti-bacterial activity is isolated from one or more high molecular weight proteins or biological agents present in the initial plasma or serum sample using ion exchange chromatography. In one embodiment, the anti-bacterial activity is isolated from one or more high molecular weight proteins or biological agents present in the initial plasma or serum sample using a single ion exchange chromatography step. In another embodiment, the anti-bacterial activity is isolated from one or more high molecular weight proteins or biological agents present in the initial plasma or serum sample using an ion exchange chromatography step in combination with one or more additional fractionation steps or methods, involving either the same or different methods.

In a particular embodiment, the anti-bacterial activity is isolated from immunoglobulin (i.e., IgG, IgM or both) using ion exchange chromatography. In another embodiment, anti-bacterial activity is isolated from two or more high molecular weight proteins present in the initial serum sample. In a particular embodiment, the anti-bacterial activity is isolated from immunoglobulin and albumin.

(iii) Affinity chromatography involves the specific interaction between one molecule in the sample and a second molecule immobilized on a stationary phase. Proteins can be used to isolate antibodies and vice versa. The affinity may be to a specific protein or a group of proteins. If the protein to be fractionated isolated is a gamma globulin, protein A is often used. Serum which contains the secreted antibodies is put through the affinity column, and the antibodies bind to the protein A attached to the column gel. Other ligands suitable for use in isolating components of blood include heparin (clotting-factor proteins), lectins (glycoproteins), antibodies (unique antigens), and enzyme inhibitors or cofactors (enzymes). For example, VLDL and LDL can be removed from a sample using antibody-based affinity chromatography (also known as immunoabsorption). Sources include, for example, Amersham-Pharmacia and Bio-Rad.

In a particular embodiment of the present invention, the anti-bacterial activity is isolated from one or more high-molecular weight proteins or biological agents present in the initial serum or plasma sample using affinity chromatography. Affinity chromatography may be used alone or in combination with other fractionation steps or methods detailed herein. In a particular embodiment, a protein G affinity column is used to deplete the sample of IgG to further isolate the anti-bacterial activity.

Methods of isolating albumin from serum involving chromatographic adsorbents and immunoaffinity methods have been reported (SatoAK et al., Biotechnol. Prog. (2002) 18, 182-192; Dockal M et al. J. Biol. Chem. (1999) 274, 29303-29310; Rothemund D et al. Proteomics (2003) 3, 279-287).

Recent advances in the study of the human proteome have led to the development of techniques to remove high abundance proteins from serum in order to isolate LMW proteins (see generally, Tirumalai R S et al. Molecular & Cellular Proteomics (2003) 2:1096-1103). Several of these methods are designed to retain LMW proteins which would otherwise be lost in the fractionation because they tend to bind to HMW proteins. These methods are considered suitable for use in isolating the anti-bacterial activity of the plasma or serum of a mammal exposed to an inoculant from the high molecular weight proteins present therein, including, for example, immunoglobulins and albumins.

Commercial affinity depletion products are available to separate albumin and immunoglobulins from serum or plasma. For example, the ProteoExtract™ Albumin/IgG Removal Kit (CalbioChem) provides highly specific and efficient depletion of albumin and IgG from plasma or serum. Depletion of albumin and IgG removes up to 75% of total serum proteins. Applied BioSystems also manufactures affinity depletion cartridges for the removal of albumin (POROS® Anti-HAS support) and IgG (POROS® Protein G cartridge) from serum (Application Note: Protemics. Affinity Depletion Cartridges for Removal of Human Serum Albumin and Immunoglobulins from Human Serum. Applied Biosystems). Using these products, greater than 99% of IgG and albumin can be removed from serum. These commercial affinity depletion products are considered suitable for use in preparing the plasma or serum fraction of the present invention.

Polyclonal antibodies can be used to deplete plasma or serum of high molecular proteins in a single step, including albumin, IgG, IgA, haptoglobin, transferrin, and antitrypsin, using liquid chromatography. Commercial sources of multi affinity removal systems include Agilent. This technique removes 85-90% of the high abundance proteins from serum. These multi-affinity removal systems are considered suitable for use in preparing the plasma or serum fraction of the present invention.

In one embodiment of the present invention, a single fractionation is sufficient to isolate the anti-bacterial activity of the plasma or serum from one or more high-molecular weight proteins or biological agents present in the initial plasma or serum sample, such as albumin and immunoglobulin. Alternatively, two or more fractionation steps can be combined. Each step may be the same basic technique (e.g., multiple ammonium sulfate precipitations) or different (e.g., precipitation with cold ethanol in combination with chromatography or heat precipitation). Any method or combination of methods suitable for isolating the anti-bacterial activity in the initial plasma or serum sample from immunoglobulin and other high molecular weight proteins or biological agents is considered suitable for use in the present invention.

In a particular embodiment, the anti-bacterial activity is isolated from immunoglobulin and albumin present in the unprocessed plasma or serum by sequential ammonium sulfate precipitation in combination with DEAE column chromatography.

According to one embodiment of the present invention, proteins with a molecular weight of greater than approximately 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 67, 68, 69, 70, 71, 72, 73, 74, 75, 76, 77, 78, 79, 80, 81, 82, 83, 84, 85, 86, 87, 88. 89, 90, 91, 92, 93, 94, 95, 96, 97, 98, 99, 100, 101, 102, 103, 104, 105, 106, 107, 108 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144 or 145 kD or above are depleted from the initial serum or plasma sample to facilitate isolation of the anti-bacterial activity.

In a particular embodiment, the serum or plasma is depleted of proteins or biological agents with a molecular weight greater than approximately 15 kD to facilitate isolation of the anti-bacterial activity.

In another embodiment, the serum or plasma is depleted of proteins or biological agents with a molecular weight greater than approximately 20 kD to facilitate isolation of the anti-bacterial activity.

In a further embodiment, the serum or plasma is depleted of proteins or biological agents with a molecular weight greater than approximately 25 kD to facilitate isolation of the anti-bacterial activity.

In a preferred embodiment, the serum or plasma is depleted of proteins or biological agents with a molecular weight greater than approximately 30 kD to facilitate isolation of the anti-bacterial activity.

In a further embodiment, the serum or plasma is depleted of proteins or biological agents with a molecular weight greater than approximately 35 kD to facilitate isolation of the anti-bacterial activity.

In another embodiment, the serum or plasma is depleted of proteins or biological agents with a molecular weight greater than approximately 40 kD to facilitate isolation of the anti-bacterial activity.

In a further embodiment, the serum or plasma fraction is depleted of proteins or biological agents with a molecular weight greater than approximately 45 kD to facilitate isolation of the anti-bacterial activity.

In a further embodiment, the serum or plasma fraction is depleted of proteins or biological agents with a molecular weight greater than approximately 50 kD to facilitate isolation of the anti-bacterial activity.

In yet another embodiment, the serum or plasma fraction is depleted of proteins or biological agents with a molecular weight of greater than approximately 9 OkD to facilitate isolation of the anti-bacterial activity.

The term “depletion” is used to indicate a reduction in the amount of a compound(s) or molecule(s) (e.g., high molecular weights proteins) in a given sample after the sample is treated according to the method of the present invention. In one embodiment of the present invention, the sample is depleted of 100%, 99%, 98%, 97%, 96%, 95%, 94%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 56%, 54%, 53%, 52%, 51%, 50%, 49-45%, 44-40%, 39-35%, 34-30%, 29-20%, 19-10%, 10%-5%, 5%-1% of the one or more high molecular weight proteins or biological agents present in the initial plasma or serum sample (e.g., immunoglobulin, serum albumin, transferrin, haptoglobin or lipoproteins).

In one embodiment of the present invention, the plasma or serum is depleted of substantially all high molecular weight proteins and biological agents. In a particular embodiment, the plasma or serum is depleted of substantially all proteins and biological agents with a molecular weight greater than approximately 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 67, 68, 69, 70, 71, 72, 73, 74, 75, 76, 77, 78, 79, 80, 81, 82, 83, 84, 85, 86, 87, 88. 89, 90, 91, 92, 93, 94, 95, 96, 97, 98, 99, 100, 101, 102, 103, 104, 105, 106, 107, 108 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144 or 145 kD present in the initial, unprocessed plasma or serum.

In one embodiment, the plasma or serum is depleted of from about 50 to about 100% of the proteins and biological agents with a molecular weight greater than approximately 10, 11, 12, 13, 14, 15, 16, 17, 18, 19, 20, 21, 22, 23,24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 67, 68, 69, 70, 71, 72, 73, 74, 75, 76, 77, 78, 79, 80, 81, 82, 83, 84, 85, 86, 87, 88. 89, 90, 91, 92, 93, 94, 95, 96, 97, 98, 99, 100, 101, 102, 103, 104, 105, 106, 107, 108 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144 or 145 kD present in the initial, unprocessed plasma or serum.

In a particular embodiment, the plasma or serum is depleted of from about 75 to about 85%, from about 85% to about 95% , or from about 95% to about 100% of the proteins and biological agents with a molecular weight greater than approximately 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 67, 68, 69, 70, 71, 72, 73, 74, 75, 76, 77, 78, 79, 80, 81, 82, 83, 84, 85, 86, 87, 88. 89, 90, 91, 92, 93, 94, 95, 96, 97, 98, 99, 100, 101, 102, 103, 104, 105, 106, 107, 108 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144 or 145 kD present in the initial, unprocessed plasma or serum.

As noted above, while the initial sample may be depleted of one or more high molecular weight proteins or biological agents to facilitate isolation of the anti-bacterial activity, the anti-bacterial activity may initially co-fractionation with one or more of the high molecular weight proteins or agents, such that the high molecular weight fraction must be further fractionated or processed to isolate the anti-bacterial activity present therein.

Transmission of infectious disease (i.e., by viruses, bacteria or parasites) remains a concern in the use of any blood or blood product such as plasma or serum. In a further embodiment of the present invention, the blood or plasma can be sterilized prior to in vivo use. Any suitable method can be used to achieve sterilization as long as the method does not alter the product in such a way as to diminish its efficacy. Non-limiting examples of sterilization techniques suitable for use with the present invention include chemicals, heat, ultraviolet radiation and photosensitizing dyes. The plasma can also be filtered to achieve sterilization. Recent advances and new strategies for the inactivation and removal of infectious agents are contemplated for use in the present invention.

In one embodiment of the present invention, the plasma is separated from blood and sterilized by repeated centrifugation and filtration. For example, the plasma is spun at approximately 32,000 rpm on a standard centrifuge. The resultant supernatant can then be transferred, preferably under sterile conditions using sterile techniques, and then suction filtered through a 0.5 micron filter. During this preparation, the sample can be kept on ice between the centrifugation and filtration steps. The plasma can then be passed over a filter, for example a filter with at least 0.2 micron pores, and then placed in an ultracentrifuge, preferably non-refrigerated, to spin at approximately 90,000 rpm for at least 20 minutes. The supernatant can then be placed in containers, preferable sterile, in an ultracentrifuge, preferably non-refrigerated, to spin at least 150,000 rpm for at least 20 minutes. After the centrifugation, the supernatant can be passed through an anhydrous filter. The plasma can be repeatedly filtered, preferably through a 0.2 micron filter and a smaller filter, such as a 0.1 micron filter. Passage through a 0.1 micron filter allows for the plasma to be deemed sterile.

(vii) Storage and Testing of the Plasma or Serum Fraction

The resulting plasma or serum preparation can be placed in small aliquots (e.g., between 2-10 cc each) and stored for later use. Proper storage conditions for plasma and serum with respect to temperature and time are well known to those skilled in the art. For example, test tubes containing small aliquots of the plasma or serum fraction can be stored at −70° C., for at least 48 hours.

After a suitable time has passed for the samples to be stored, such as 48 hours, individual aliquots can be brought to room temperature for sterility testing. For example, the sample can be cultured under both anaerobic and aerobic conditions to test for contamination. If the cultures are negative, the remaining aliquots of can then are administered to a patient.

(viii) Administration of the Plasma or Serum Fraction to Patient in Need Thereof

The plasma or serum fraction can be administered to a patient in need thereof through any means provided in this application (See Pharmaceutical Compositions below). In one embodiment, the patient can receive a therapeutically effective dosage, preferably between if administered subcutaneously, and treatment duration can vary based on the severity of the bacterial infection.

B. Gram Positive Bacterial Infections and Related Conditions

The plasma or serum fraction of the present invention can be used to treat or prevent infections caused by Gram- positive bacteria. It can be used to treat infections caused by any of the Gram-positive bacteria mentioned in the Background of the Invention, as well as the following non-limiting examples of clinical relevance that follow.

The present invention can be used to treat or prevent infection with Gram positive cocci (aerobic and anaerobic) including members of the Staphylococcus genus (e.g., Staphylococcus aureus, Staphylococcus epidermidis, S. haemolyticus, S. hominis, S. exotoxin and S. saprophyticus); the Streptococcus genus (e.g., Streptococcus pyogenes, Streptococcus pneumoniae, Streptococcus agalactia, Streptococcus mutans); the Entenococcus genus (e.g., E. faecium, E. faecalis, E. avium, E. casseliflavus, E. durans, E. gallinarum, E. dispar, E. hirae, E. flavescens, E. mundtii, E. solitanius, E. raffinosus); Peptostreptococcus sp. (e.g. Peptostreptococcus magnus, Peptostreptococcus asaccharolyticus, Peptostreptococcus anaerobius, Peptostreptococcus prevotii, and Peptostreptococcus micros.); Veillonella; catalase-negative gram-positive cocci including viridans streptococcal species such as S. mutans and S. sobrinus, S. salivanius and S. vestibularis, S. bovis, S. pneumoniae, S. sanguis and S. gordonii, S. mitis and S. oralis, S. anginosus, S. constellatus, and S. intermedius, S. milleni, S. MG-intermedius, S. anginosus-constellatus; Abiotrophia and Granulicatella; the Gemella genus (e.g., Gemella haemolysans, Gemella morbillorum, Gemella bergeniae, Gemella sanguinis); Rothia mucilaginosa; Aerococcus (e.g., Aerococcus vinidans, A. uninae); Lactococcus (e.g., L. lactis, L.s garviae); Helcococcus (e.g., Helcococcus kunzii); the genus Globicatella (e.g., Globicatella sanguis); Facklamia; Ignavigranum; Dolosicoccus; Dolosigranulum (e.g., Dolosigranulum pigrum); Alloiococcus (e.g., A. otitidis); Vagococcus (e.g., V. fluvialis and V. salmoninarum); Leuconostoc (e.g., L. citreum, L. lactis, L. mesenteroides, L. pseudomesenteroides, L. argentinum and L. paramesenteroides); Pediococcus (e.g., P. acidilactici and P. pentosaceus), Tetragenococcus (e.g., Tetragenococcus halophilus), as well as other clinically-relevant Gram-positive cocci well known in the art.

The present invention can also be used to treat or prevent infections caused by Gram-positive bacilli including, Lactobacillus sp., Clostnidium sp. (Clostnidium botulinum, Clostridium botulinum, Clostnidium perfringens, Clostnidium tetani); Actinomyces sp.(e.g., A. Israeli), Bifidobactenium (e.g., B. dentium), Nocardia sp, Listenia monocytogenes, Corynebactenium diptheniae, Propionibactenium acnes; Bacillus anthracis, and Erysipelothnix rhusiopathiae, among others well known in the art.

Diseases and symptoms caused by Gram-positive bacteria are varied and well known to clinicians, including the following non-limiting examples: pneumonia, meningitis, sepsis, bacterial endocarditis, streptococcal exudative pharyngitis, cellulitis (“flesh-eating infection”), wound infection, and visceral abscesses, acute rheumatic fever, poststreptococcal glomerulonephritis, urinary tract infections, septicemia, bacteremia, osteomyelitis, appendicitis, otitis media, colon cancer, strep throat, scarlet fever, impetigo, sinusitis, peritonitis, arthritis, strep pneumonia or pneumococcal pneumonia, pharyngitis, tonsillitis, mastoiditis, joint and bone infections, erysipelas, chorioamnionitis, endometri endometritis, skin and soft tissue infection, conjunctivitis, enterocolitis, toxic shock syndrome peritonitis, among many others.

C. Gram Negative Bacterial Infections and Related Conditions

The plasma or serum fraction of the present invention can be used to treat Gram negative bacterial infections. It can be used to treat any of the infections caused by Gram-negative bacteria mentioned in the Background of the Invention, as well as the specific, non-limiting examples of Gram-negative bacteria that follow.

The present invention can be used to treat or prevent infections caused by Gram-negative bacilli including the Klebsiella (e.g., K pneumoniae); Citrobacter; Serratia; Enterobacter; Proteus (P. mirabilis, P. vulganis, and P. myxofaciens); Morganella (e.g., M morganiz); Providencia (P. rettgeni, P. alcalifaciens, and P. stuartil); Salmonella sp. (e.g., S. lyphi, S. paratyphi A, B S. schottmuelleni, S. hirschfeldii, S. entenitidis); Salmonella sp. (e.g., S. entenitidis S. typhimunium, S. heidelberg, S. newport, S. infantis, S. agona, S. montevideo, and S. saint-paul); the Shigella genus (e.g., S. flexneni, S. sonnei, S. boydii, S. dysenteniae); the Haemophilus genus (e.g., H. influenzae); Brucella sp. (e.g., Brucella abortus, B. melitensis, B. suis, B. canis); Francisella tularensis; Vibnio sp. (e.g., V. cholerae, V. parahaemolyticus, V. mimicus, V. alginolyticus, V. hollisae, V. vulnificus); Yersinia sp. (e.g., Y. pestis, Y. enterocolitica); Bunkholdenia sp. (e.g., B. pseudomallei, B. cepacia); Campylobacter sp. (e.g., C. fetus, C. jejuni, C. coli); Helicobacter pylon; Serratia marcescens; Acinetobacter baumannii, Actinobacillus actinomycetemcomitans, Bordetella pertussis; Capnocytophaga; Cardiobactenium hominis; Eikenella corrodens; Kingella kingii; Legionella pneumophila; Pasteurella multisided; Acinetobacter; Xanthomonas malt ophilia; Aeromonas; Plesiomonas shigelloides, as well as many others known in the art.

The present invention can also be used to treat or prevent infections caused by Gram-negative cocci including, without limitation, Neissenia sp.(e.g., N. gonorrhoeae and N. meningitides), Moraxella (Branhamella) catarrhalis, Veillonella sp. (e.g., Veillonella parvula) and Acinetobacter sp.

Diseases and symptoms caused by Gram-negative bacteria are also well known to clinicians and include the following non-limiting examples: diarrhea; urinary tract infections, hepatobiliary, peritoneal, cutaneous, and pulmonary infections; ear infections, mastoid sinus infections, bacteremia, pharyngitis, fever, headache, pharyngitis, constipation, anorexia, abdominal pain and tenderness, dysuria, nonproductive cough, epistaxis, splenomegaly, leucopenia, anemia, liver function abnormalities, proteinuri, acute cholecystitis, hepatitis, pneumonia, osteomyelitis, endocarditis, meningitis, soft tissue abscesses, glomerulitis, gastroenteritis, septicemia, endocarditis, conjunctivitis, epiglottitis, bacterernic Brazilian purpuric fever, chancroid, encephalitis, neuritis, orchitis, cholecystitis, hepatic suppuration, mediastinitis, lung abscess, cholera, hypovolemia renal tubular necrosis, plague, meliodosis, bronchitis, endocarditis, cellulites, arthritis, sexually transmitted diseases, urethritis, cervicitis, proctitis, salpingitis, epididymitis, skin and bone lesions, and typhoid fever, among many others.

The composition and method of the present invention can also be used to treat bacteria for which Gram stain is not applicable (i.e., that fall outside of the Gram-negative or Gram-positive designation). These include, for example, Borrelia sp. (e.g., Borrelia recurrentis, B hernsii and B tunicatae); Bartonella sp., which cause Cat scratch fever, Oroya fever, bacillary angiomatosis, among other diseases; Chlamydiae (e.g., C trachomatis, C psittaci, and C pneumoniae); Calymmatobactenium granulomatis; Leptospira sp. (e.g., Leptospira interrogans); Rickettsia sp. Treponema sp. (e.g., Treponemapallidum), and others known in the art.

Experiments with whole serum or plasma derived from a goat exposed to an HIV-bearing inoculant, not otherwise processed or depleted high molecular weight proteins, suggests that a whole serum composition of that type may not be effective against E. coli and Pseudomonas aeruginosa (data not shown). However, that composition differs from the composition of the present invention.

D. Other Agents That Can Be Used in Combination and/or Alternation With the Composition of the Present Invention

The plasma or serum fraction can be administered alone or can be administered in combination or alternation with other agents/drugs that can be used to treat or prevent Gram-positive bacteria infections. It can also be used alone or in combination or alteration with other agents or drugs used to treat or prevent Gram-negative bacterial infections.

In general, during alternation therapy, an effective dosage of each agent is administered serially, whereas in combination therapy, effective dosages of two or more agents are administered together. The dosages will depend on such factors as absorption, bio-distribution, metabolism and excretion rates for each drug/agent/composition as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions. Examples of suitable dosage ranges can be found in the scientific literature and in the Physicians Desk Reference. Many examples of suitable dosage ranges for other compounds described herein are also found in public literature or can be identified using known procedures. These dosage ranges can be modified as desired to achieve a desired result.

The plasma or serum fraction of the present invention can also be used in combination and/or alternation with any of the Gram-positive bacterial infection treatments described in the Background of the Invention of this specification, or any agents listed below. Non-limiting examples include:
Beta lactams (e.g., penicillin G, ampicillin) and/or antipseudomonal penicillins
Penicillinase-resistant penicillins (e.g., methicillin, oxacillin, nafcillin, cloxacillin, dicloxacillin)
Cephalosporins (e.g., cephalothin, cefazolin, cephalexin, cephradine, cefamandole, cefoxitin, and 3rd-generation cephalosporins);
Carbapenems (e.g., imipenem, meropenem, Biapenem)
Gentamicin
Streptogramins (e.g., Quinupristin/dalfopristun (Synercid™))
Streptomycin
Ceftriaxone
Cefotaxime
Rifampin
Glycopeptides (e.g. vancomycin, teicoplanin, LY-333328 (Ortivancin))
Macrolides (e.g., erythromycin, clarithromycin, azithromycin, lincomycin, and clindamycun)
Ketolides (e.g., Telithromycin, ABT-773)
Tetracyclines
Glycylcyclines (e.g., Terbutyl-minocycline (GAR-936))
Aminoglycosides
Bacitracin
Chloramphenicol
Imipenem-cilastatin
Glycopeptides (e.g., oritavancin, LY-333328, dalbavancin)
Quinolones
Fluoroquinolones (e.g., ofloxacin, sparfioxacin, gemifloxacin, cinafloxacun (DU-6859a)) and other topoisomerase inhibitors
Trimethoprim-sulfamethoxazole (TMP-SMX)
Cloxacillin
Dicloxacillin
Ciprofloxacin
topical mupirocin
Oxazolidinones (e.g., AZD-2563, Linezolid (Zyvox™))
Lipopeptides (e.g., Daptomycin, Ramoplanin)
ARBELIC (TD-6424) (Theravance)
TD-6424 (Theravance)

For the treatment of TB, the composition of the present invention can be used alone or in combination or alteration with drugs for the treatment of TB, including MDR-TB, including without limitation, isoniazid (INN), rifampin (RIF), pyrazinamide (PZA), streptomycin (SM), Ethambutol (EMB), Capreomycin, cycloserine, ethionamide (ETH), kanamycun, and p-aminosalicylic acid (PAS). The composition may also be used to treat TB in combination or alternation with any of the following drugs: pyridones and quunolizines; ascidimine compounds; third generation macrolides; quinolones (e.g., KRQ-10018); isoniazid analogs (e.g., MJH-98-1-81), nitroimazidopyrans (e.g., PA-9647, PA 822,PA 824); rifalazil analogs; nitroimidazoles (e.g.)2-ethyl-5-intro-2,3-dihydro imidazo-oxazole; pyrroles (e.g., L3858); moxifloxican; fluoroquinoles (Biyskier A, Lowther J. “Fluoroquinolones and tuberculosis” Expert Opin Investig Drugs. 2002 11(2):233-58) including ofloxacin (Floxin), levofloxacin (L-ofloxacin) the optically active isomer of ofloxacin, ciprofloxacin (Cipro), sparfioxacin (SPFX) (AT-4 140), AM-1155 and lomefloxacin; macrolides including clarithromycin (Biaxin) and azithromycin (Zithromax); inhibitors of beta-lactamase and new beta-lactamase-resistant antibiotics; rifamycins such as Rifabutin (RBT; Mycobutin); bactericidal aminoglycosides (e.g., amikacin, capreomycin, kanamycin, paromomycin); fusidic acid; thiacetazone, rifabutin, rifapentine, KRM-1648, pyrazunamide, kanamycin, amikacun, capreomycun, gentamicin, tobramycin, ethambutol, para-aminosalicylic acid, D-cycloserine, ofloxacun, levofloxacin, ciprofloxacin, sparfloin, clofazimine, clarithromycin, azithromycun, erythromycin, cefoxitin, cefinetazole, imipenem, sulfamethoxazole, sulfisoxazole, sulfadiazune, sulfathiazole, trimethoprim, and doxycycline.

The plasma or serum fraction of the present invention can also be used in combination and/or alternation with any of the Gram(−) negative bacterial infection treatments described in the Background of the Invention of this specification, or any agent listed below. Non-limiting examples include:
Ampicillin
Tetracyclines
Broad spectrum penicillins (e.g., ticarcillin, piperacillin)
Cephalosporins
Aminoglycosides
Trimethoprim-sulfamethoxazole (TMP-SMX)
Quinolones
Carbenicillin
Ceftriaxone
Cefoperazone
Chloramphenicol
Glucocorticoids (e.g., prednisone, dexamethasone)
Ciprofloxacin
Doxycycline
Streptomycin
Rifampin
Gentamicin
Furazolidone
Erythromycun
Norfioxacin
Tobramycin
Polymyxin B
Colistin
Mezlocillin
Azlocillin
Aztreonam
Imipenem
Meropenem
Ciprofloxacin
Piperacillin
Ticarcillin
Fluoroquinolones
Peptides (e.g., baceteriacidal permeability inducing peptide (BPI), MSI-78, nisin)
   E. Adjuvants that Can Be Used in Combination and/or Alternation With the Composition of the Present Invention

In addition, if desired, the plasma or serum fraction may contain auxiliary substances such as wetting or emulsifying agents, pH buffering agents or adjuvants that enhance the effectiveness of the vaccines. As non-limiting examples, the plasma or serum fraction can be administered in combination or alternation with any of the following known adjuvants.

Adjumer (PCPP salt; polyphosphazene; polyidi(carboxylatophenoxy)lphosphazene) which may be administered in the soluble form as an adjuvant for parenteral formulations or in the crosslinked form as a microsphere hydrogel for mucosal formulations. It induces a sustained antibody response after a single parenteral immunization and these antibody responses include antigen specific IgG1 and IgG2a. with sustained IgG and IgA responses also induced in after mucosal immunization. Algal Glucan (also known as β-glucan or glucan) is administered with antigen for enhancement of both humoral and cell-mediated immunity. β-Glucans exert their immunostimulatory activities by binding to specific β-glucan receptors on macrophages. This ligand-receptor interaction results in macrophage activation and, in certain formulations, promotes antigen targeting. Algammulun (gamma inulin/alum composite adjuvant) is used in formulations as a primary adjuvant and stimulates immune responses by causing ligation of leukocyte-surface complement receptors (CR) via known biochemical mechanisms, thus placing the antigen close to activated leukocytes. Addition of Algammulin is known to enhance both humoral and cell-mediated immunity from either Th1 or Th2 pathways, depending on the weight ratio of inulin to Alhydrogel. Avridine (N,N-dioctadecyl-N′,N′-bis(2-hydroxyethyl)propanediamine; CP20,961) may be incorporated into a liposomal preparation; into aqueous suspensions from alcoholic solution; in Intralipid, an aqueous soybean oil emulsion vehicle; other vegetable and mineral oil vehicles; in Tween 80 dispersions in saline; in saline suspension with alum-precipitated antigen. It has been shown to cause humoral and cellular immunity, proliferation of B and T lymphocytes, protective immunity, activation of macrophages, induction of interferon, enhancement of mucosal immunity when administered orally/enterically with antigen, adjuvanticity with a variety of antigens, induction of IgG2a and IgG2b isotypes. BAY R1005 (N-(2-Deoxy-2-L-leucylamino-β-D-glucopyranosyl)-N-octadecyldodecanoylamide hydroacetate) can be used as a primary adjuvant. BAY R1005 in combination with purified virus vaccines or subunit vaccines led to increased protection of virus-challenged mice. The increase in antibody synthesis induced by BAY R1005 is specifically dependent on the antigen and it acts on the proliferation of B lymphocytes as a second signal which has no effect until the antigen acts as a first signal. BAY R 1005 is capable of activating B lymphocytes without the helper function of T lymphocytes. In mice parenteral immunization with recombinant urease mixed with BAY R1005 induced strong Th1 and Th2 responses and thereby elicited better protection against Helicobacter pylori infection than adjuvants which induced a prominent Th2 type response only (Guy, B., et al., 1998. Systemic immunization with urease protects mice against Helicobacten pylon infection. Vaccine 16:850-856.) Calcitriol (1α,25-dihydroxyvitamin D3; 1,25-di(OH)₂D₃; 1,25-DHCC; 1α,25-dihydroxycholecalciferol; 9,10-seco(5Z,7E)-5,7,10(19)-cholestatriene-1α,3β,25-triol) has been shown to promote the induction of mucosal and systemic immunity when incorporated into vaccine formulations. Calcium Phosphate Gel has been used as adjuvant in vaccine formulations against diphtheria, tetanus, pertussis and poliomyelitis. It adsorbs soluble antigens and presents them in a particulate form to the immune system and contains no components that are not natural constituents of the body and is very well tolerated. Cholera toxin B subunit (CTB, also known as CTB subunit) augments humoral responses by acting as an efficient carrier/delivery system and is completely non-toxic and has been used extensively in humans without negative side-effects. Cholera holotoxin (CT) has been shown to augment both humoral and cell-mediated immunity, including CTL responses, and thereby enhances MHC class I and II restricted responses. CT exerts immunomodulating effects on T cells, B cells as well as antigen-presenting cells (APC). Cholera toxin Al-subunit-ProteinA D-fragment fusion protein (CTA1-DD gene fusion protein) has proven equivalently potent as an adjuvant to the intact cholera holotoxin (CT) for humoral and cell-mediated immunity. CTA1-DD is targeted to B lymphocytes, both memory and naive cells and acts as a powerful systemic and mucosal adjuvant.

Block Copolymer P1205 (CRL1005) acts as both an adjuvant and stabilizer and forms microparticulate structures that can bind a variety of antigens via a combination of hydrophobic interactions and surface charge. Cytokine-containing Dehydration Rehydration Vesicles (Cytokine-containing Liposomes) induces both cellular and humoral immunity. Dimethyldioctadecylammonium bromide; dimethyldistearylammonium bromide (DDA—CAS Registry Number 3700-67-2) is known for stimulation immune responses against various antigens and especially delayed type hypersensitivity. DHEA (Dehydroepiandrosterone; 5-androsten-3β-ol-17-one; dehydroisoandrosterone; androstenolone; prasterone; transdehydroandrosterone; DHA) can be directly incorporated into vaccine formulations and will enhance antibody formation. DHEA can be administered systemically at the time of vaccination, or can be directly incorporated into the vaccine formulation. DMPC (Dimyristoyl phosphatidyl choline; sn-3-phosphatidyl choline-1,2-dimyristoyl; 1,2-dimyristoyl-sn-3-phosphatidyl choline; (CAS Registry Number 18194-24-6)) and DMPG (Dimyristoyl phosphatidylglycerol; sn-3-phosphatidyl glycerol-1,2-dimyristoyl, sodium salt (CAS Registry Number 67232-80-8); 1,2-dimyristoyl-sn-3-phosphatidyl glycerol) are used in the manufacture of pharmaceutical grade liposomes, typically in combination with DMPG and/or cholesterol and are also used in adjuvant systems for vaccine formulations. DOC/Alum Complex (Deoxycholic Acid Sodium Salt; DOC/Al(OH)₃/mineral carrier complex) is a complex used as adjuvant formulation and is known to enhance the immune response to membrane proteins. Freund's Complete Adjuvant is a mixture of mineral oil (Marco 52) and emulsifier (Arlacel A [mannide monooleate]) as an emulsion of 85% mineral oil and 15% emulsifier with heat-killed antigen. Gamma Inulin is a highly specific activator of the alternative pathway of complement in vitro and in vivo included in adjuvant formulations as a primary adjuvant and also as the immune stimulant when combined as composite particles with alum in the adjuvant Algammulin. It is expected that it stimulates immune responses by causing ligation of leukocyte-surface complement receptors (CR) via known biochemical mechanisms. Addition of gamma inulin is known to enhance both humoral and cell-mediated immunity from both Th1 and Th2 pathways. Gamma inulin also has an antitumor action and an effect on natural immunity. Gerbu Adjuvant, an adjuvant based on GMDP with DDA and Zinc-L-proline have been shown to complex as synergists. GM-CSF (Granulocyte-macrophage colony stimulating factor; Sargramostim (yeast-derived rh-GM-CSF)) is a glycoprotein of 127 amino acids and recombinant human GM-CSF is produced in yeast and it differs from the natural human GM-CSF by substitution of Leu for Arg at position 23. This cytokine is a growth factor that stimulates non-nal myeloid precursors, and activates mature granulocytes and macrophages.

GMDP (N-acetylglucosaminyl-(β1-4)-N-acetylmuramyl-L-alanyl-D-isoglutamine (CAS Registry Number 70280-03-4)—Semi-synthetic. Disaccharide isolated from microbial origin, dipeptide wholly synthetic. U.S. Pat. No. 4,395,399) is known as a primary adjuvant. It has been shown to be an highly effective primary adjuvant in a range of vehicles; aqueous buffers, mineral oil, pluronic/squalane/Tween emulsions. Also effective as oral adjuvant, enhancing mucosal IgA response. Imiquimod (1-(2-methypropyl)-IH-imidazo[4,5-c]quinolin-4-amine; R-837; S26308) can be included in adjuvant formulations as a primary adjuvant component and is known to induce both humoral and cell-mediated immunity via induction of cytokines from monocytes and macrophages. ImmTher™ (N-acetylglucosaminyl-N-acetyhnuramyl-L-Ala-D-isoGlu-L-Ala glycerol dipalmitate; DTP-GDP) is a potent macrophage activator which induces high levels of TNF, IL-1, and IL-6 both in vitro and in vivo (U.S. Pat. No. 4,950,645). Immunoliposomes prepared from Dehydration-Rehyrdation Vesicles (DRVs) (Immunoliposomes Containing Antibodies to Costimulatory Molecules) are composed of phosphotidyicholine/cholesterol/biotinylate d-phospotidylethanolamine (PC/CH/PEB) in a molar ration of 5:5: 1. Antigen is added to the water suspension of DRV followed by repeated vortexing and lyophylization of the liposome suspension. Interferon-γ (Actimmune® (rhIFN-gamma, Genentech, Inc.); immune interferon; IFN-γ; gamma-interferon) has demonstrated higher and earlier neutralizing antibody titers, an increase in duration of neutralizing antibody titers, an increase in MHC class 11 expression on antigen presenting cells, increase in Helper T cell levels, and an improved DTH response. The IFN-gamma is preferably given at the same site and at the same time (within 6 hrs) as the antigen. Interleukin-1β (IL-10; IL-1; human Interleukin 1β mature polypeptide 117-259) is known as a primary adjuvant and is active by oral, intravenous, intraperitoneal and subcutaneous routes. It increases both T-dependent and T-independent responses to different types of antigens and can be active in both primary and secondary responses. Interleukin-2 (IL-2; T-cell growth factor; aldesleukin (des-alanyl-1, serine-125 human interleukin 2); Proleukin®; Teceleukin®) is used as a primary adjuvant, co-emulsified with antigens and lipids, with polyethylene glycol modified long acting form (PEG IL-2), or liposome encapsulated sustained release dosage form. IL-2 supports the growth and proliferation of antigen-activated T lymphocytes and plays a central role in the cascade of cellular events involved in the immune response. Proliferating T-cells also produce a variety of other lymphokines which may modulate other arms of the immune system and in view of these direct and indirect actions of IL-2 on the immune response, IL-2 functions as an adjuvant to vaccination by increasing the specific and durable response to vaccine immunogens. Low doses may give up to 25-fold increase in adjuvant effect, with inhibition of adjuvant effect at high doses. May induce cellular immunity when given systemically, and IgA when administered at a mucosal surface. Interleukin-7 (IL-7) has been shown to enhance antibody production as a primary adjuvant in liposome formulated sustained release form (Bui, et al. “Effect of MTP-PE liposomes and interleukin-7 on induction of antibody and cell-mediated immune responses to a recombinant HIV-envelope protein”, J Acquired Immune Deficiency Syndrome, 1994 August;7(8):799-806.) and has also been used co-emulsified with antigen and lipids. Interleukin-12 (IL-12; natural killer cell stimulatory factor (NKSF); cytotoxic lymphocyte maturation factor (CLMF)) is used as a primary adjuvant component to enhance Thi-dependent cell-mediated immune responses including cytolytic T-lymphocyte responses.

Immune stimulating complexes (ISCOM(s)™) are a complex composed of typically Quillaja saponins, cholesterol, phospholipid, and antigen in phosphate-buffered saline (PBS). They are antigen-presenting structures that have been shown to generate long-lasting biologically functional antibody response. ISCOMs have demonstrated a protective immunity and a functional cell-mediated immune response, including Class I restricted CTLs have been reported in several systems. They have generally been administered subcutaneously or intramuscularly but non-parenteral administrations (intranasal and oral) have also proven to be effective. Liposomes (L) containing protein or Th-cell and/or B-cell peptides, or microbes with or without co-entrapped interieukin-2, BisHOP or DOTMA A, [L (Antigen)]; B, [L (IL-2 or DOTMA or BisHOP+Antigen)]; C, [L (Antigen)-mannose]; D, [L (Th-cell and B-cell epitopes)]; E, [L (microbes)] act as carrier of Th-cell peptide antigen which provides help for co-entrapped B-cell antigen to overcome genetic restriction and induce immunological memory. They may also act as carriers of attenuated or live microbial vaccines to deliver microbes and co-entrapped soluble antigens or cytokines simultaneously to antigen-presenting cells or to protect entrapped vaccines from interaction with maternal antibodies or antibodies to vaccine impurities in preimmunized subjects. Loxoribine (7-allyl-8-oxoguanosine) is known as a primary adjuvant for antibody responses to a wide variety of antigen types in a variety of species. It augments CTL-mediated, NK cell-mediated, macrophage mediated, and LAK cell-mediated cytotoxicity, induces IFN(a/b/γ, TNΦa, TNΦb, IL-1a, IL-6 and up regulates humoral immune responses in immunodeficiency. LT-OA or LT Oral Adjuvant induces both mucosal and systemic immunity (both humoral [including IgA and IgG2, isotypes] and cell-mediated) to killed microorganisms or peptide antigens mixed with it in neutral non-phosphate buffered saline, with/without sodium bicarbonate. MF59 (Squalene/water emulsion—composition: 43 mg/mL squalene, 2.5 mg/mL polyoxyethylene sorbitan monooleate (Polysorbate 80), 2.4 mg/mL sorbitan trioleate (Span 85)) in combination with a variety of subunit antigens results in elevated humoral response, increase T cell proliferation and presence of cytotoxic lymphocytes. MONTANIDE ISA 51 (Purified IFA; Incomplete Freund's adjuvant) addition induces humoral and cell-mediated immunity with various antigens. MONTANIDE ISA 720 (metabolizable oil adjuvant) induces humoral and cell-mediated immunity with various antigens. MPL™ (3-Q-desacyl-4′-monophosphoryl lipid A; 3D-MLA) is used as a primary adjuvant in adjuvant formulations. Its activity is manifested either alone in aqueous solution with antigen, or in combination with particulate vehicles (e.g., oil-in-water emulsions) and its activity may be enhanced by use of vehicle that enforces close association with antigen. MTP-PE (N-acetyl-L-alanyl-D-isoglutaminyl-L-alanine-2-(1,2-dipalmitoyl-sn-glycero-3-(hydroxy˜phosphoryloxy))ethylamide, mono sodium salt) and alternately MTP-PE liposomes are optionally a part of MF59 and are known as immunomodulators. The addition of MTP-PE to the MF59-based HIV vaccine in HIV seropositive individuals resulted in a marked increase in HIV antigen lymphocyte proliferation. Murametide (Nac-Mur-L-Ala-D-Gln-OCH3) induces granulocytosis and enhances the humoral response. Murametide displays the same profile of adjuvant activity as MDP and has been chosen for development because of its favorable therapeutic ratio. When administered in 50% water-in-oil emulsion, it mimics the activity of Freund's complete adjuvant without its side effects (U.S. Pat. No. 4,693,998.) Murapalmitine (Nac-Mur-L-Thr-D-isoGIn-sn-glycerol dipalmitoyl) is administered in water-in-oil emulsion as an adjuvant of humoral and cell-mediated responses. D-Murapalmitune (Nac-Mur-D-Ala-D-isoGln-sn-glycerol dipalmitoyl) is a strong adjuvant of humoral and cell-mediated immunity when administered in a 50% mineral oil emulsion. NAGO is a mixture of the two enzymes-neuraminidase and galactose oxidase Ag 1:5 ratio in units of activity. It generates cell surface Schiff base-forming aldehydes on antigen presenting cells and Th-cells, thereby amplifying physiologic Schiff base formation that occurs between cell-surface ligands as an essential element in APC:T-cell inductive interaction. It is a potent non-inflammatory adjuvant with viral, bacterial and protozoal subunit vaccines, and is especially effective in the generation of cytotoxic T-cells.

Non-Ionic Surfactant Vesicles (NISV) induces both a humoral and cell-mediated immune response and preferentially stimulates the Th1 sub-population of T-helper cells. It is known to be effective with antigens within a broad size range, from short peptides to particulates, and has extremely low toxicity. Pleuran (β-glucan; glucan) has shown in experimental studies that rabbits as well as mice immunized once by coadministration of viral antigens and 60 μg of Pleuran produced at least 20-fold higher antibody titers than control animals injected with the immunogen alone (Chihara, G. et al., 1989, Lentinan as a host defense potentiator (HDP), Int. J. Immunother. 4:145-154.) PLGA, PGA, and PLA (Homo-and co-polymers of lactic and glycolic acid; Lactide/glycolide polymers; poly-lactic-co-glycolide) used in vaccine delivery have demonstrated an ability to control the release of antigen after administration, thereby eliminating or reducing the need for boost immunizations. Antigens incorporated in PLGA microspheres have exhibited enhanced and prolonged antibody activity responses compared to equivalent doses of free antigen. Pluronic L121 (poloxamer 401) enhances the presentation of antigen to cells of the immune system. PMMA (polymethyl methacrylate) is known as a primary adjuvant for all types of antigens. PODDS™ (proteinoid microspheres) serves as a vehicle for oral immunization, protecting the antigen and allowing for co-encapsulation of adjuvants with antigens in microspheres. Poly rA:Poly rU (a double helix comprised of polyadenylic acid and polyuridylic acid) is known as an adjuvant to humoral and cell-mediated immunity when given with antigen; it increases non-specific immunity to microorganisms. Polysorbate 80 may be used in emulsion vaccine formulations including MF59, SAF-1 and Antigen Formulation. Protein cochleates act as both carriers and adjuvants, providing multivalent presentation of antigens to the immune system, with maintenance of native conformation and biological activity and providing protection of antigens from degradation following oral delivery. They stimulate strong mucosal and systemic antibody, proliferative and cytotoxic responses to associated antigens. QS-21 (Stimulon™ QS-21 Adjuvant) can be used in vaccine formulations as a primary adjuvant component for enhancement of both humoral and cell-mediated immunity. Quil-A (Quil-A saponin, Quillaja saponin) is used in veterinary vaccines and for production of ISCOMs. Rehydragel HPA (High Protein Adsorbency Aluminum Hydroxide Gel; alum) and Rehydragel LV (low viscosity alluminurn hydroxide gel; alum) are primary adjuvants in parenteral vaccine formulations and aluminum compounds (aluminum hydroxide, aluminum phosphate, alum) are currently the only vaccine adjuvants used in US-licensed vaccines. The use of aluminum adjuvants are accompanied by stimulation of IL-4 and stimulation of the T-helper-2 subsets in mice, with enhanced IgGi and IgE production. S-28463 (4-Amino-otec,-dimethyl-2.. ethoxymethyl-lH-imidazo[4,5-c]quinoline 1-ethanol) induces both humoral and cell-mediated immunity via induction of cytokines from monocytes and macrophages. Experimental results indicate S-28463 is about 100-fold more potent than imiquimod in antiviral models and in cytokine induction from monocytes and macrophages. Syntex Adjuvant Formulation (SAF, SAF-1, SAF-m) causes antigens to arrange on the surface of the emulsion droplets partly because of their amphipathic nature, and partly because of hydrogen bonding with poloxamer 401. The emulsion droplets also activate complement, as demonstrated by consumption of C3 and production of C3b; the latter, on the surface of droplets, targets them to antigen-presenting cells (follicular dendritic cells and interdigitatung cells) in lymph nodes of the drainage chain and possibly in more distant lymphoid tissues. In this way the emulsion facilitates the presentation of antigens to responding lymphocytes (threonyl-MDP monograph.) Sclavo peptide (IL-β3 163-171 peptide) enhances immune response to T-dependent and T-independent antigens. It is known as a primary adjuvant and may be administered i.p, i.v., s.c. or p.o and it is active either when administered separately from antigen, or admixed with antigen, or physically linked to antigen. Sendai Proteoliposomes, Sendai-containing Lipid Matrices (Sendai glycoprotein15 containing vesicles; fusogenic proteoliposomes; FPLs; Sendai lipid matrix-based vaccines) are potent immunogens and have the ability to stimulate strong T helper and CD8+ cytotoxic T cell responses (CTL) to lipid bilayer-integrated glycoproteins as well as encapsulated peptides, proteins and whole formalin-fixed viruses. These vesicles also act as effective delivery vehicles for drugs and proteins.

Span 85 (Arlacel 85, sorbitan trioleate) is used as an emulsification agent in MF59 adjuvant formulation. Specol (Marcol 52 (mineral oil, paraffins, and cycloparaffins, chain length 13-22 C atoms) Span 85 (emulsifier, sorbitan trioleate) Tween 85 (emulsifier, polyoxyethylene-20-trioleate)) all are individually FDA approved for veterinary use and they function as a depot (slow release of antigen) and a polyclonal activator (independent of presence of antigen) for cells of the immune system (cytokine release). Squalane (Spinacane;Robane®;2,6,10,15,19,23-hexamethyltetracosane) is a component of Antigen Formulation (AF) and Syntex Adjuvant Formulation (SAF), and constitutes the oil component of the emulsion. Stearyl Tyrosine (octadecyl tyrosine hydrochloride) has adjuvanticity similar to aluminum hydroxide with bacterial vaccines; superior to aluminum hydroxide with viral vaccines. Theramide™ (N-acetyloglucosaminyl-N-acetylinuramyl-L-Ala-D-isoGlu-L-Ala-dipalmitoxy propylamide (DTP-DPP)) is a potent macrophage activator and adjuvant. It induces IL-6, IL-12, TNF, IFN-γ, and relatively lessor quantities of IL-10. The compound preferentially induces cellular immunity. Threonyl-MDP (Termurtide™; [thr 1]-MDP; N-acetyl muramyl-L-threonyl-D-isoglutamine) induces the production of a cascade of cytokines, including IL-1a, IL-iβ and IL-6. Responding lymphocytes release IL-2 and IFN-γ and the latter increases the production of antibodies of certain isotypes, including IgG2a. This isotype, and the homologous IgGi in primates, interacts with high affinity Fcγ receptors, so that the antibodies can function efficiently in opsonizing viruses and other infectious agents for uptake by phagocytic cells. Ty Particles or Ty Virus-Like Particles present antigen in a polyvalent, particulate form. Cytotoxic T-lymphocytes are induced in the absence of any other adjuvant formulations. Walter Reed Liposomes (Liposomes containing lipid A adsorbed to aluminum hydroxide, [L(Lipid A+Antigen)+Alum]) have been shown to induce both humoral and cell-mediated immunity. Liposomes containing lipid A provide a very potent adjuvant activity. Absorption of liposomes containing lipid A to aluminum hydroxide gel contributes additional strong adjuvant activity.

F. Pharmaceutical Compositions

Subjects, such as humans, suffering from a bacterial infection(s) or related conditions can be treated by administering to the subject in need thereof an effective amount of the defined plasma or serum fraction in the presence of a pharmaceutically acceptable carrier or diluent. The fraction can be administered by any appropriate route, for example, orally, parenterally, enterally, intravenously, intradermally, subcutaneously, topically, nasally, rectally, in liquid, or solid form.

The plasma or serum fraction is included in the pharmaceutically acceptable carrier or diluent in an amount sufficient to deliver to a patient a therapeutically effective amount to reduce symptoms without causing serious side effects in the treated patient

It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The plasma or serum fraction may be administered at once, or may be divided into a number of smaller doses to be administered at varying intervals of time.

A preferred mode of administration of the active compound is through subcutaneous injection, which can optionally include an inert diluent or carrier. Preferred carriers are physiological saline, phosphate buffered saline (PBS) or Ringer's Solution.

If orally administered, the plasma or serum fraction will be lyophilized and will generally include an inert diluent or an edible carrier. It may be enclosed in gelatin capsules or compressed into tablets. For the purpose of oral therapeutic administration, the plasma or serum fraction can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as algiic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The plasma or serum fraction can also be mixed with other active materials that do not impair the desired action, or with materials that supplement the desired action, as discussed in more detail above. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic.

If administered by nasal aerosol or inhalation, the plasma or serum fraction is prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

If rectally or vaginally administered in the form of suppositories, the plasma or serum fraction may be prepared by mixing the drug with a suitable non-initiating excipient, such as cocoa butter, synthetic glyceride esters of polyethylene glycols, which are solid at ordinary temperatures, but liquefy and/or dissolve in the rectal cavity to release the drug.

In a preferred embodiment, the plasma or serum fraction is prepared with carriers that will protect it against rapid elimination from the body, such as a controlled release formulation, including implants and micro-encapsulated delivery systems. Biodegradable, biocompatible polymers can be used, such as ethylene vinyl acetate, polyanhydrides, polyglycolic acid, collagen, polyorthoesters and polylactic acid. Methods for preparation of such formulations will be apparent to those skilled in the art. The materials can also be obtained commercially from Alza Corporation.

Liposomal suspensions (including liposomes targeted to infected cells with monoclonal antibodies to viral antigens) are also preferred as pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. The plasma or serum fraction is then introduced into the container. The container is then swirled by hand to free lipid material from the sides of the container and to disperse lipid aggregates, thereby forming the liposomal suspension.

### EXAMPLES

Experiments have been conducted to prepare the serum fraction of the present invention, and are conducted in vitro to show that the composition of the present invention can inhibit Gram-positive and Gram-negative bacteria. Results are summarized below.

### Example 1

### Production of the Inoculant

Patients who are HIV positive and with a detectable viral load, and preferably with a viral load above 2,000, were used for blood samples. The blood taken from the patient was centrifuged at 32,000 rpm at room temperature using standard sterile laboratory techniques and the resulting patient plasma/serum is frozen at −20° C. The sample was frozen no more than 24 hours, but can be left longer.

### Example 2

### Inoculation of the Animal

The animal used in the process was first inspected by a veterinarian and evaluated for any underlying abnormalities in the animal and for any pathogens that may cause a possible zoonosis. Once the animal was found to be healthy it was then well maintained in a clean environment and monitored by a veterinarian on a regular basis.

The HIV+patient plasma sample prepared as outlined in Example 1 was allowed to thaw to room temperature and approximately 3 cc of the patient plasma/serum was injected subcutaneously into the animal according to standard sterile procedures. Once the animal was been injected with the sample, the animal is carefully marked and labeled assigning it a number and indicating where the sample was taken and from whom. A three week period of time was allowed to pass prior to harvesting any of the animal's blood.

### Example 4

### Antibody Depletion

Antibody depletion experiments were conducted on the product prepared as described in Example 3 by the panning technique as follows. Experiments were completed to remove IgG, 1gM and both IgG and 1gM. Two wells each of a polystyrene high protein-binding flat-bottomed plate were coated with 100 μL of 1 μg/mL of anti-goat IgG, anti-goat 1gM, anti-goat IgG+antigoat 1gM antibodies, or PBS. The plate was incubated for 3 hours at room temperature. Following the incubation the antibodies and PBS were removed and 200 μL of blocking solution (0.05% Tween 20, 10 mg/mL BSA in PBS) was added and the plate was incubated overnight at 4° C. Following the overnight incubation, the wells were washed 5 times with wash buffer (PBS plus 0.05% Tween-20). One hundred fifty microliters (150 μL) of the serum product was added to each of the wells and the plate was incubated for 2 hours at 37° C.

The serum was then removed and tested in bacterial growth inhibition assays. The ability of the antibody-depleted serum to inhibit the growth of various bacteria is assessed in nutrient growth broth cultures. In general, bacteria are inoculated to nutrient media used for growth of each microorganism and the amount of bacterial growth is quantified after a well defined period of time in the presence or absence of various dilutions of the antibody-depleted serum ranging from 1:20 through 1:20,480. Growth in the presence of the antibody-depleted serum is compared directly to the growth of the bacteria in nutrient broth and whole serum (i.e., serum not subject to antibody depletion) to quantify the inhibition of bacterial growth by the antibody-depleted serum.

In all cases, complete anti-bacterial activity could be observed following the treatments, indicating that the removal of goat immunoglobulins (IgG, 1gM and IgG+1gM) may not impact the anti-bacterial activity of the test material. Preliminary heat denaturation could further suggest that active component(s) is not an antibody. Incubation of the antibody-depleted serum product at 56° C. for 30 mm could result in inactivation of specific anti-bacterial activity, while the same treatment of control goat serum might not reduce the level of an observed non-specific inhibition (data not shown).

### Example 5

### Anti-Bacterial Activity of the Initial Plasma or Serum Sample

The anti-bacterial activity of the initial plasma or serum sample is used as a baseline for determining the efficiency of the concentration of the anti-bacterial activity for the fractionation procedures described below.

The anti-bacterial activity of the initial serum sample is determined by: (1) measuring the protein concentration of the serum fraction using Bradford or Lowery based method for determining protein concentration; (2) determining the activity of the fraction using established in vitro using an anti-bacterial assay (as described above in Example 4); (3) assigning a unit of activity to the fraction based on the amount of protein needed to achieve a 50% inhibition of bacterial growth (IC50). This unit can be used as a baseline to track the fold purification obtained through the fractionation process.

### Example 6

### Anti-HIV Activity of the Initial Plasma or Serum Sample

The anti-HIV activity of the initial plasma or serum sample was used as a baseline for determining the efficiency of the concentration of the anti-HIV activity for the fractionation procedures described below.

The anti-HIV activity of the initial serum sample is determined by: (1) measuring the protein concentration of the serum fraction using Bradford or Lowery based method for determining protein concentration; (2) determining the activity of the fraction using established in vitro anti-viral assay (attachment assay); (3) assigning a unit of activity to the fraction based on the amount of protein needed to achieve a 50% inhibition of cell proliferation (IC50). This unit can be used as a baseline to track the fold purification obtained through the fractionation process.

### Example 7

### Ammonium Sulfate Precipitation

Immunoglobulin G (IgG) is depleted from the sample using a 33% ammonium sulfate precipitation. With the serum sample kept on ice and with constant slow stirring, a saturated ammonium sulfate solution (e.g., 450 g of ammonium sulfate in water to 500 mL) is added to 33% v/v (1 ml saturated ammonium sulfate per 2 mL of serum). The sample is allowed to stir on ice for a period of time ranging from approximately 2 to approximately 4 hours, and then centrifuged for approximately 12,000×g for approximately 20 minutes at 4° C. The supernatant is carefully removed to a clean tube. The precipitate or pellet should contain the majority of the IgG.

The pellet is then washed twice with a volume of ice cold 33% ammonium sulfate solution equivalent to the original volume of the fraction, and then centrifuged at approximately 12,000×g for 20 minutes at 4° C. for each wash. The pellet is then dissolved in a volume of ice cold buffer A equivalent to 10% of the starting volume. The buffer should be suitable for in vitro assays and down stream purification procedures. The suspended pellet is then desalted using a desalting column or dialyzed overnight in ice cold buffer at 4° C. in order to remove any ammonium sulfate.

If a desalting column is used, the procedure involves decanting buffer from the top of 20 column, and loading the sample onto the column. Then, 10 ml of Buffer A is applied to the top of the column and allow to flow through the column. The sample (no more than 3 mL) should be applied to the top of the column and allowed to pass through the column by gravity flow. Fractions of 0.5 ml volume into siliconized tubes.

Then, the 33% ammonium sulfate supernatant produced is fractionated with a 66% 25 ammonium sulfate precipitation. The concentration of the supernatant is adjusted to 66% by adding 1 ml of saturated ammoium sulfate for every 1 ml of supernatant. The 66% precipitation is then performed as above with respect to the 33% precipitation above to provide a 66% pellet. The 66% pellet is then washed twice with a volume of ice cold 66% ammonium sulfate solution equivalent to the original volume of the fraction, and then centrifuged at approximately 12,000×g for 20 min 4° C. for each wash. The pellet is then dissolved in a volume of ice cold buffer A to 10% of the original fraction volume. The suspended pellet is desalted using either a desalting column or by dialysis against buffer A at 4° C. overnight to remove any ammonium sulfate.

Next, the activity of each fraction is tested. The protein concentration of each fraction is measured using the BioRad protein assay. An bacterial growth assay is performed using a concentration of protein equivalent to or less than the amount of protein from the unfractionated material that yielded 50% inhibition in the initial assay. A unit of activity is assigned to the fraction based on the amount of protein and the initial dilution needed to achieve a 50% inhibition of bacterial growth (IC50). The fold purification is determined by calculating the ratio of activity of the purified material to the starting material. The fold purification is determined by calculating the ratio of the activity of the purified material to the activity of the starting material, as described in Example 5. This unit should be calculated as activity per mass of total protein and should increase as the purification process is applied.

An analysis of the active fraction or fractions is then performed. Native and denatured PAGE is performed to determine the approximate number and sizes of the proteins in the active fraction(s). An immunoblot analysis is performed to test for the presence of immunoglobulins and albumin in the active fraction.

### Example 8

### DEA Affi-Blue Column Chromatography

DEA Blue Econo-Pac cartridges (BioRad) are used for initial fractionation of the serum sample. These reagents contain an affinity matrix of Cibacron blue dye coupled with a DEAE anion exchanger. The Cibacron blue dye has a high affinity for protein albumins and the DEAE allows for the separation of proteins based on their charge. Immunoglobulins do not bind to the Cibacron blue dye, albumuns will bind very tightly, and other proteins should have low to intermediate binding capacity. The various proteins within the serum will also have a range of DEAE binding capacities. The low to intermediate Cibacron blue and the DEAE binding proteins can be eluted from the matrix using competing salt ions. Elution can be performed using a step gradient (as outlined below) or with a linear gradient. The procedure outlined herein details the use of a syringe to load the protein and buffers onto the column; however, the procedure may also be adapted for use with a low or high pressure chromatography system and a larger chromatography column.

(i) Preparation of Buffers

The various buffers needed are prepared. The equilibration and wash buffer (“Buffer A”) contains 28 mM NaCl and 20 mM Tris-HC1 pH 8.0. The elution buffers (“Buffer E”) for the DEAE blue cartridge include E1, E2, ES and Ei4. E1 is 100 mM NaC1, 20 mM Tris-HC1 pH8.0; E2 is 250 mM NaCl 20 mM Tris-HC1 pH 8.0; ES is 500 mM NaCl 20 mM Tris-HC1 pH 8.0; E14 is 1.4M NaC1 20 mM Tris-HC1, pH 8.0. Regeneration buffer 1 (Buffer G) is 1.4 M NaCl 100 mM Acetic Acid pH 3.0 40% Isopropanol. Regeneration buffer 2 (“Buffer I”) is 28 mM NaC1 20 mM Tris-HC1 2M Guanidine-HC1. Storage Buffer for DEAE Blue cartridge is 20 mM Sodium Phosphate pH 7.5 0.02% Sodium Azide. Necessary additives for protein stabilization or activity (such as protease inhibitors, glycerol, EDTA, DTT, etc.) may be added to any of the buffers as deemed necessary. The pH listed for the buffers should be the pH at 25° C. All buffers should be chilled to 0-8° C. prior to use to minimize loss of anti-viral activity.

(ii) Sample Preparation

The sample should be in Buffer A. If the starting material is not already in Buffer A, equilibrate the sample with buffer A using a desalting column or by dialysis.

(iii) Preparing the Cartridge for Use

The cartridge is prepared for use by washing it with 10 ml of Buffer G at a flow rate of 1 ml/min to remove any residual dye. It is then washed with 5 ml of buffer El 4 at a flow rate of 2 ml/min. A small amount of air may remain just above the upper Mt and in the inlet nozzle of the cartridge. The cartridge should be inverted so that the arrow points upward, allowing air to be expelled into the cartridge and out through the outlet nozzle. The cartridge is then washed with Buffer A for 10 minutes at a flow rate of 2.0 ml/mm. The cartridge should then be equilibrated with Buffer A for 2 mm at 1.0 ml/mm. The cartridge should then be inverted, so it points downward.

(iv) Chromatography

The following procedures are used when a syringe is used to load and elute the protein onto the column. (A chromatography system utilizes the same buffers, but they are applied with the system pump and the protein is eluted with a linear salt gradient rather than a step gradient). To load the sample, a sterile syringe is pre-west with Buffer A by sucking and expelling buffer into and out of the syringe. The plunger is removed from the syringe and attached to the cartridge using a luer lock connector. The equilibrated sample is then added to the barrel of the syringe. The plunger is inserted, and the sample is pushed through the cartridge taking care not to inject air into the cartridge. The flow through into a clean siliconized tube is collected as the sample is being loaded. The flow through can be collected into more than one tube.

The cartridge is then washed. The syringe is removed from the cartridge, and washed with buffer A. The plunger is pulled from the syringe and re-attached the barrel to the cartridge.

The barrel is then filled with Buffer A and pushed through the cartridge at a flow rate of about 1 ml/mm. Wash fractions of ˜0.5 ml are then collected in siliconized tubes. Wash with a total volume equivalent to 3-5 times the cartridge volume.

The bound proteins are then eluted with a step gradient. The syringe is removed from the cartridge and washed with Buffer El. The syringe is then attached to the cartridge and pushed through 10 to 15 ml of buffer El. The fractions are collected in siliconized tubes. The elution steps are then repeated with E-Buffer's of increasing NaCl concentrations. Alternatively, a linear salt eradient from 0 to 500 mM NaCl can be used to elute the bound serum proteins.

The sample is then analyzed. The concentration of protein in each sample is analyzed using the Bio-Rad Protein assay. The peak fractions are analyzed for anti-bacterial activity in vitro. The protein profile of the fractions is analyzed on a polyacrylamide gel. The presence of 1gG and 1gM by are detected by western blot.

### Example 9

### Protein G Affinity Chromatography

Protein G is a cell surface protein of group G streptococci. It is a Type III F_{c} receptor that binds to the F_{c} region of TgG by a non-immune mechanism. Protein G binds tightly to different subclasses of 1gG from a variety of species including human, rabbit, horse, sheep, and goat. IgG immunoglobulins are removed from active fractions using Protein G coupled to a Sepharose matrix. The IgG binds tightly to the matrix and the active non-immunoglobulun fraction will not bind and will be collected in the flow though during application.

An Amersharn HiTrap Protein G HP column with a syringe for application of the sample and buffer can be used for Protein G affinity chromatography. The buffers are prepared, including a binding buffer (20 mM Sodium Phosphate, pH 7.0) and an elution buffer (0.1M Glycine-HC1, pH 2.7). Necessary additives for protein stabilization or activity (such as protease inhibitors, glycerol, EDTA, DTT, etc.) may be added to any of the buffers as deemed necessary. The pH listed for the buffers should be the pH at 25° C. All buffers should be chilled to 0-8° C. prior to use to minimize loss of anti-viral activity.

The sample is prepared by adjusting to the composition of Protein G binding buffer using either a desalting column or by dialysis. Use of a desalting column involves decanting buffer from the top of column, and loading the sample onto the column. Then, 10 ml of Buffer A is applied to the top of the column and allow to flow through the column. No more than 3 ml sample should be applied to the top of the column and allowed to pass through the column by gravity flow. Fractions of 0.5 ml volume into siliconized tubes.

If the sample is cloudy or viscous, the buffer adjusted sample is passed through a 0.45 μm filter prior to loading. The column is then prepared. Silicon collected tubes are prepared for the collection of eluted IgG by adding 60-200 1 of 1M Tris-HC1 pH 9.0 per ml of fraction collected. Tris should not be added to tubes for collection of flow through material. Using a syringe or pump, the column is washed with 10 column volumes of binding buffer.

The sample is then applied to the column and the flow through is collected in siliconized tubes. The flow through will contain the non-IgG proteins. The flow through is collected in 0.5 to 1 mL fractions. The column is washed with 5-10 column volumes of binding buffer. The washed fractions are then collected in siliconized tubes. The bound IgG is then eluted with 2-5 column volumes of elution buffer. The eluted protein material is collected in siliconized tubes containing 1M Tris-HC1, pH 9.0.

The sample is then analyzed The protein concentration in each sample is quantified using the Bio-Rad Protein assay. The peak fractions are analyzed for anti-bacterial activity in vitro. The protein profile of the fractions is analyzed on a polyacrylamide gel. The fractions are also analyzed for the presence of IgG and 1gM by western blot.

### Example 10

### Gel Filtration Chromatography

Gel filtration chromatography is a method of separating molecules based on their size. This procedure may be used at any step in the purification process. Immunoglobulins and serum albumins will fractionate with the higher molecular weight proteins (in the first eluted fractions to come off of the column) and lower molecular weight proteins, such as cytokines, will come off of the column in the latter fractions. Different gel filtration media can be used for separation purposes depending on the size of the protein to be isolated.

The gel filtration media can be Sephacryl SiOO or S200 FIR. The sample is prepared by adjusting the composition of the gel filtration buffer using either a desalting column or by dialysis. The desalting protocol involves decanting buffer from the top of column, and loading the sample onto the column. Then, 10 ml of Buffer A from Example 7 is applied to the top of the column and allow to flow through the column. No more than 3 ml sample should be applied to the top of the column and allowed to pass through the column by gravity flow. Fractions of 0.5 ml volume into siliconized tubes. If necessary, the sample can be concentrated using a Centricon concentrator (or comparable). If the sample is cloudy or viscous, the buffer-adjusted sample is passed through a 0.45 μm filter prior to loading.

Using a chromatography system, the column is properly attached to the system and equilibrated with gel filtration buffer. chromatography system, properly attach the column to the system and equilibrate the column with GF-buffer (0.05 M Sodium phosphate buffer, pH 7.4, 0.15 M NaCl). The sample is loaded on to the column through the sample loop. Proteins are then eluted with gel filtration buffer at a constant flow rate. The fractions are collected in siliconized tubes.

The samples are analyzed for anti-bacterial activity in vitro. The samples are further 15 analyzed for protein profile on a polyacrylamide gel. The samples are also analyzed for the presence of IgG and 1gM by western blot.

### Example 11

### Preparation of the Product Using an HIV-Bearing Inoculant

Pathogen-free goats were inoculated with plasma from an uninfected human donor, and plasma from an HIV-infected human donor. Blood was extracted from the goats just prior to inoculation (Week 0) and at weekly intervals up to 5 weeks post inoculation (including a 3 week interval). Serum samples were prepared from the goat blood.

### Example 12

### Preparation of the Product Using a Gram-Positive Bacterial Inoculant and a Gram-Negative Bacterial Inoculant

Pathogen-free goats are inoculated with plasma from an uninfected human donor, plasma from Staphylococcus aureus infected human donor and plasma from an E. coli infected donor. Blood is extracted from the goats just prior to inoculation (Week 0) and at weekly intervals up to 5 weeks post inoculation. Serum samples were prepared from the goat blood.

### Example 13

### Partial Fractionation of the Product

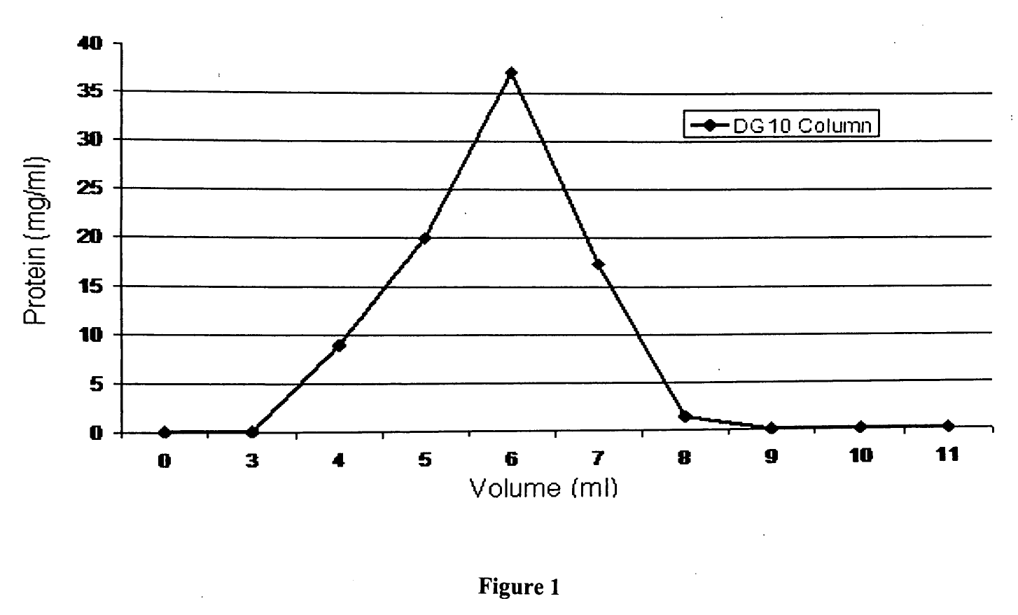

A serum fraction prepared as described in Example 11 was subject to partial fractionation. Specifically, serum was collected at 3 week from a goat inoculated as described in Example 10 (i.e., with 5 ml of plasma from an HIV infected individual). Specifically, serum was collected from animal number 26. The serum was equilibrated with Buffer A (10 mM Tris-HC1 pH 8.0, 28 mM NaCl) using a Bio Rad DG-10 gravity flow column at 4° C. Fractions of approximately 1 ml each were collected by hand and analyzed for protein content using the Bio Rad Protein Assay with BSA as a reference standard. The elution profile from the desalting column is shown in FIG. 1.

The fractions for milliliters 4 through 8 from the DG-10 column were pooled and subjected to DEAE-blue chromatography using a Bio Rad 5 ml DEAE-blue cartridge and 20 ml syringe. Sample was loaded onto the cartridge with the syringe and the cartridge was washed with 25 ml of ice cold buffer A (fractions 1 to 35), followed by sequential washing with ice cold buffer A containing 0.1M NaCl (fractions 36 to 50), ice cold buffer A containing 0.5 M NaCl (fractions Si to 66), and a final elution with ice cold buffer A containing 1.4 M NaCl (fractions 67 to 77). All fractions were collected manually and immediately placed on ice. The protein concentration in each fraction was determined using the Bio Rad Protein assay.

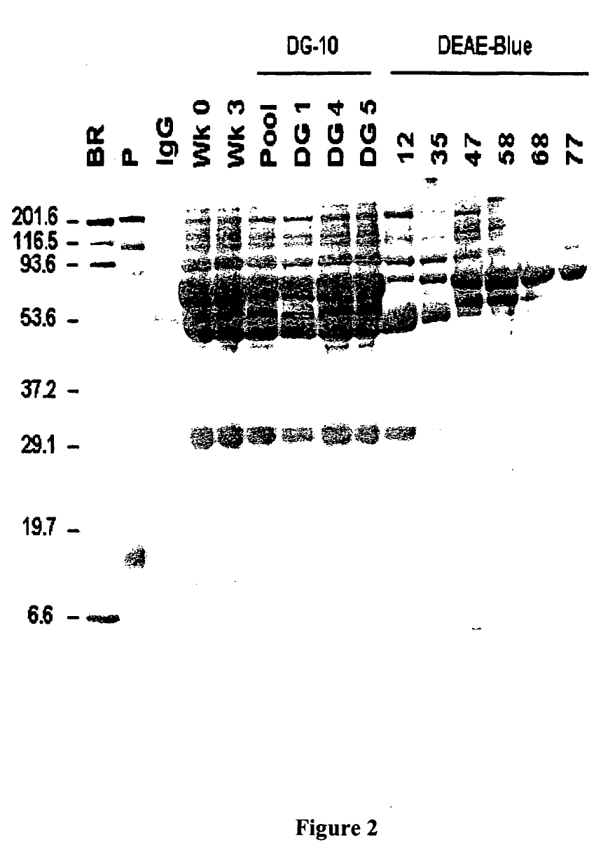

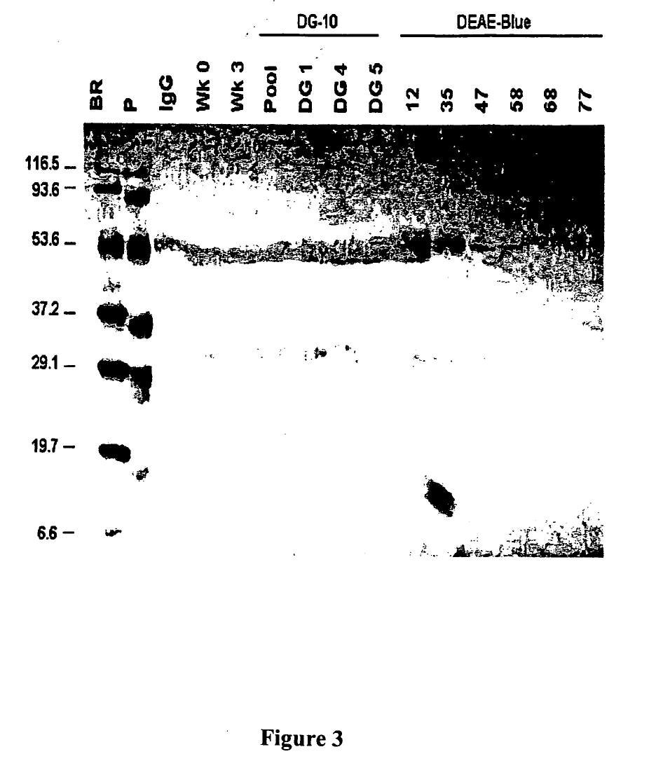

Selected fractions were subjected to SDS-PAGE on 8-16% Bio Rad Criterion gels followed by Coomassie blue staining (FIG. 2) and immunoblot analysis with horse radish peroxidase conjugated rabbit-anti-goat IgG polyclonal antisera (FIG. 3). The following amounts of total protein were loaded onto the gels for both the Coomassie stain and immunoblot gels, 20 μg of total protein for Week 0 serum, Week 3 serum, and DG-iO column fractions (pool, DG-b, DG-4, and DG-5); 10 μg of total protein for DEAE-blue fractions 12, 47, 57, and 68; 5 μg of total protein for DEAE-blue fraction 35; 2.5 μg of total protein for DEAE-blue fraction 77; and 2 μg of total purified total goat IgG (NIH AIDS Research and Reference Reagent Program). Proteins for immunoblot analysis were transferred to 0.45 micron nitrocellulose using a Bio Rad semi-dry transfer apparatus. The membrane was blocked overnight at 4° C. with 5% nonfat milk in PBS-T (PBS plus 0.1% Tween-20) and probed with a 1:2000 dilution of horse radish peroxidase conjugated rabbit-anti-goat IgG polyclonal antibody in PBS-T plus 1% nonfat milk for 1.5 hours at room temperature. Following probing, the membrane was washed extensively with PBS-T and developed with One-step TMB HRP-detection reagent (Pierce) according to the manufacturers instructions.

The serum samples described in Examples 12 are subject to partial fractionation and analysis, as described above for the Example 10 serum sample.

### Example 14

### In Vitro Results

The fractionated compositions prepared above in Example 13 (i.e., the fractionated product of serum samples prepared as described in Examples 11 and 12) are evaluated against bacteria in vitro in order to evaluate the anti-bacterial activity, including bacterial growth assays for both gram-positive and gram-negative bacteria assays. Results could suggest that the high molecular weight protein-depleted fractions from goats challenged with HIV, Staphylococcus aureus and E. coli possess anti-bacterial activity.

### Example 15

### Partial Fractionation of the Product

The serum fraction prepared as described in Examples 11 was subject to partial fractionation. Specifically, a total of 88 mls of the blood from animal number 26 were removed from the freezer and allowed to thaw on ice.

(i) Ammonium Sulfate Precipitation

The serum sample was divided into 4 polypropylene centrifuge tubes each containing a flea stir bar and placed in an ice bath. A 33% ammonium sulfate precipitation was performed by adding one part of ice cold saturated ammonium sulfate solution per 2 parts serum in 0.5 ml aliquots to each tube with constant stirring. The mixture was allowed to stir on ice for 1.5 hr. Following the 1.5 hrs, the mixture was subjected to centrifugation at 12,000×g for 20 minutes 4° C. The supernatant was transferred to a 400 ml polypropylene beaker and subjected to 66% ammonium sulfate precipitation, as above, with the addition of 1 part saturated ammonium sulfate solution per 1 part supernatant. The pellets from the 33% ammonium sulfate precipitation were washed with 33% ammonium sulfate in PBS and then suspended in 20 mls PBS. The supernatant from the 66% ammonium sulfate precipitation was decanted into 50 ml conical tubes and the pellet was suspended in 20 ml PBS.

The results of the precipitation procedure are shown below in Table I:

TABLE I

| | Volume | Conc. | Total Protein | |
|---|---|---|---|---|
| Fraction | (ml) | (mg/ml) | (mg) | % Protein |
| Initial Bleed Out | 88 | 79.85 | 7026.6 | 100 |
| 33% Wash | 81 | 40.12 | 325 | 4.62 |
| 33% Pellet | 23 | 38.45 | 884.3 | 12.58 |
| 66% Pellet | 39.6 | 88.46 | 3503.1 | 49.85 |
| 66% Sup. | 193.2 | 43.81 | 846.5 | 12.05 |

(ii) DEA-Blue Column Chromatography

Two milliliters of a partially fractionated serum sample previously subjected to 66% ammonium sulfate precipitation and dialysis was further fractionated on a 5 ml BioRad DEAE Blue column using a BioRad Biologic LP chromatography system.

A DEAE-Blue Econo column (BioRad) was prepared according to the manufacturer's instruction. Briefly, using a syringe, the column was washed with 10 ml of freshly prepared Buffer R (1.4 M NaCI, 0.1M Acetic acid, 40% Isopropanol) followed by washings with 5 ml Buffer B (1.4 M NaC1, 20 mM Tris-HC1 pH 7.5, 10% Glycerol) and 30 ml of Buffer A (28 mM NaCl, 20 mM Tris-HC1 pH 7.5, 10% Glycerol). The column was then attached to the BioRad BioLogic LP Chromatography system and equilibrated with Buffer A (approximately 50 ml at 1 ml/min). Two milliliters of a dialyzed 66% ammonium sulfate pellet fraction prepared above was 25 thawed on ice and loaded onto the BioLogic LP using a 3 ml syringe and a 2 ml sample loop. The sample was loaded onto the column by running 6 mls of Buffer A at a flow rate of 1 ml/mm through the sample loop, and the column was washed with eight column volumes (40 ml) of Buffer A at the same flow rate. The bound proteins were eluted using a linear salt gradient from 28 mM NaCl to 1.4 mM NaC1 over 40 mls at 1 ml/min. Eighty two fractions of approximately 0.75 ml each were collected into siliconized 1.5 ml microcentrifuge tubes. Chromatography was monitored using the LP Data View software package. All fractions were stored at −80 C until analyzed. Samples were analyzed for protein content using the BioRad Protein assay kit with BSA as a quantitative standard. The majority of protein was bound to the column and eluted with the linear gradient; a minority of protein came through in the unbound flow through.

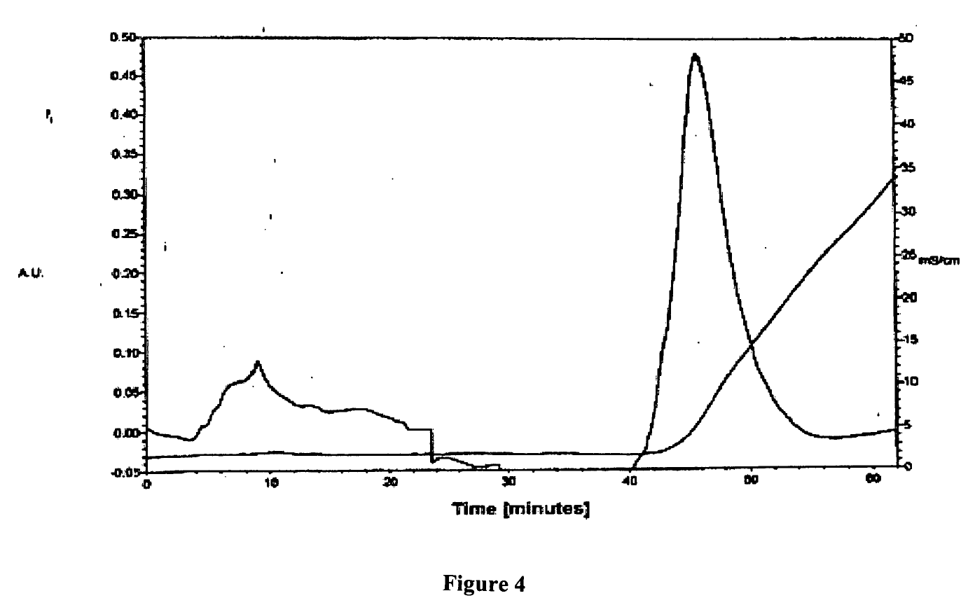

The chromatographic profile of the DEAE-Blue column fractionation of the 66% ammonium sulfate pellet is shown in FIG. 4. Detection of protein occurred four minutes into the run at fraction 6. The concentration of protein in the flow through fractions peaked between 9 and 10 minutes into the run (fraction 13) and then declined until around 26 minutes (fraction 36). The majority of protein in the flow through fractions (concentrations greater than 100 ng/p.l) was contained in fractions 9 through 20. A linear salt gradient was initiated 40 minutes into the run and the majority of total protein was eluted between 40 and 54 minutes (fractions 56 to 74). Based on the chromatographic profile, this gradient did not appear to selectively fractionate proteins based on ionic charge.

The serum fraction prepared as described in Examples 11 and 12 are subject to fractionation, as described above.

(iii) SDS-PAGE

Selected fractions of the serum fraction prepared as described in Example 10 and fractionated as described above were also analyzed by SDS-PAGE. Five micrograms of total protein from selected fractions, including the initial bleed out serum, the dialyzed 66% ammoium sulfate pellet, and DEAE-blue fractions 11-15, and 57-77, were prepared in 2× Lammeli sample buffer, heated to 95° C. for 5 mm, and loaded onto a Criterion Precast 8-16% polyacrylamide Tris-HCI gel (Bio Rad). The gel was also loaded with Bio Rad prestained broad range molecular weight markers. The proteins were electrophoresed at 100 V until the bomophenol dye in the sample buffer reached the bottom of the gel. The gel was subjected to Coomassie blue staining with BioSafe Coomassie G250 stain (Bio Rad) according to the manufacturers recommended method. An image of the stained gel was captured on an Alphalmager 2000 and the same software was used to calculate molecular weight of the fractionated proteins.

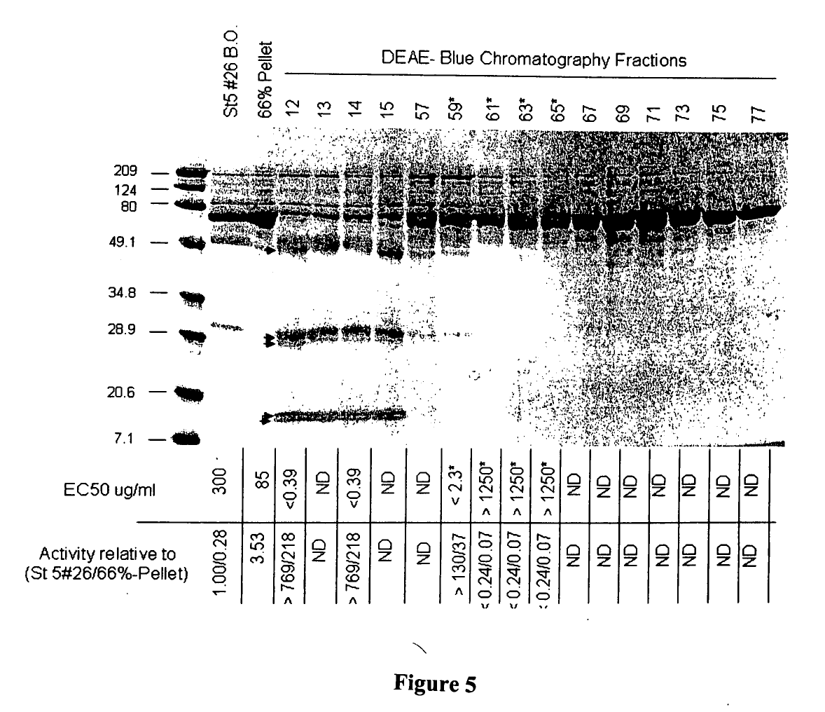

SDS-PAGE analysis and Commassie staining revealed five proteins appear enriched in the unbound fractions. As indicated by the arrow in FIG. 5, five proteins appear enriched in fractions 11 through 15, although other proteins may be present that are not detected by the staining technique. These proteins are (from the top) 49.1, 30.1, 28.6, 14.i, and 12.2 kDa in molecular weight. Light staining bands of equivalent size are found in fractions 57, 59, and 61, but not in fractions 63 through 77.

Selected fractions of the serum fraction prepared as described in Example 12 are fractionated as described above are also analyzed by SDS page. Results could suggest several low molecular weight proteins enriched in the unbound fractions.

### Example 16

### In Vitro Results

A crude serum sample dialyzed against Buffer A, the dialyzed 66% ammonium sulfate pellet, and DEAE-Blue fractions 12, 14, 60, 62, 64, and 66 is analyzed for anti-bacterial activity in vitro (growth assays for gram-positive and gram-negative bacteria). The results could suggest that the fractions prepared as described in Examples 11 and 12 exhibit anti-bacterial activity.

The invention has been described with reference to its preferred embodiments. Variations and modifications of the invention will be obvious to those skilled in the art from the forgoing detailed description of the invention.

What is claimed is:

1. A plasma or serum fraction useful in the treatment or prevention of a bacterial infection or related condition, which fraction is derived from a mammal exposed to an inoculant, and which fraction has been depleted of one or more high molecular weight proteins or biological agents present in the unprocessed plasma or serum.

2. The plasma or serum fraction of claim 1, wherein the inoculant is a bacteria-bearing inoculant.

3. The plasma or serum fraction of claim 2, wherein the bacteria-bearing inoculant is the .he blood, plasma or serum of a subject infected with bacteria.

4. The plasma or serum fraction of claim 2, wherein the bacteria-bearing inoculant is a bacterial lysate, or tissue from a subject infected with bacteria.

5. The plasma or serum fraction of claim 2, wherein the bacteria-bearing inoculant is a lysate of a cysts or other inclusion body containing bacteria, a purified bacterial preparation grown in vitro, or a suspension of bacteria in saline, plasma or another biological fluid.

6. The plasma or serum fraction of claim 2, wherein the bacterial bearing inoculant is a Gram-positive bacteria-bearing inoculant.

7. The plasma or serum fraction of claim 6, wherein the Gram-positive bacteria-bearing inoculant is selected from the group consisting of Staphylococcus-bearing inoculants, Streptococcus-bearing inoculants and Enterococcus-bearing inoculants.

8. The plasma or serum fraction of claim 7, wherein the Staphylococcus-bearing inoculant is a Staphylococcus aureus-bearing inoculant.

9. The plasma or serum fraction of claim 8, wherein the Staphylococcus aureus-bearing inoculant is a methicillin-resistant Staphylococcus aureus-bearing inoculant.

10. The plasma or serum fraction of claim 7, wherein the Streptococcus-bearing inoculant is a Streptococcus pneumonia-bearing inoculant.

11. The plasma or serum fraction of claim 10, wherein the Streptococcus pneumonia-bearing inoculant inoculant is a penicillin resistant Streptococcus pneumonia-bearing inoculant.

12. The plasma or serum fraction of claim 7, wherein the Enterococcus-bearing inoculant is a vancomycin-resistant Enterococcus-bearing inoculant.

13. The plasma or serum fraction of claim 2, wherein the bacteria-bearing inoculant is a Gram-negative bacteria-bearing inoculant selected from the group comprising Salmonella, Shigella, Escherichia, Klebsiella, Enterobacter, Serratia, Proteus, Morganella, Providencia, Yersinia, Neisseria, Moraxella (Branhamella), and Acinetobacter-bearing inoculants.

14. The plasma or serum fraction of claim 1, wherein the inoculant is an HIV-bearing inoculant.

15. The plasma or serum fraction of claim 1, wherein the plasma or serum fraction is depleted of approximately 1-10%, 10-20%, 20-30%, 30-40%, 40-50%, 50-60%, 60-70%, 70-80%, 80-90%, or 90-100% of the one or more high molecular weight proteins present in the unprocessed plasma or serum sample.

16. The plasma or serum fraction of claim 1, wherein the plasma or serum fraction has been depleted of immunoglobulin present in the unprocessed plasma or serum.

17. The plasma or serum fraction of claim 16, wherein the plasma or serum fraction has been depleted of approximately -10%, 10-20%, 20-30%, 30-40%, 40-50%, 50-60%, 60-70%, 70-80%, 80-90%, or 90-100% of the immunoglobulin present in the unprocessed plasma or serum sample.

18. The plasma or serum fraction of claim 1, wherein the bacterial infection is a Gram-positive bacterial infection.

19. The plasma or serum fraction of claim 18, wherein the Gram-positive bacterial infection is selected from the group consisting of Staphylococcus infections, Streptococcus infections and Enterococcus infections.

20. The plasma or serum fraction of claim 1, wherein the bacterial infection is a Gram-negative bacterial infection.

21. A plasma or serum fraction useful for the treatment and prevention of a bacterial infection and related conditions, which fraction is derived from a mammal exposed to an inoculant, and which fraction has been depleted of two or more high molecular weight proteins or biological agents present in the unprocessed plasma or serum.

22. The plasma or serum fraction of claim 21, wherein the plasma or serum fraction has been depleted of immunoglobulin and albumin present in the unprocessed plasma or serum sample.

23. A plasma or serum fraction useful for the treatment or prevention of a bacterial infection and related conditions which fraction is derived from a mammal exposed to an inoculant, and which fraction has been depleted of proteins or biological agents with a molecular weight greater than approximately 50 kD present in the unprocessed plasma or serum sample.

24. The plasma or serum fraction of claim 23, wherein the inoculant is a bacteria-bearing inoculant.

25. The plasma or serum fraction of claim 23, wherein the inoculant is a viral-bearing inoculant.

26. A plasma or serum fraction useful for the treatment or prevention of a bacterial infection which fraction is derived from a mammal exposed to an inoculant, and which fraction has been depleted of proteins or biological agents with a molecular weight greater than approximately 30 kD present in the unprocessed plasma or serum sample.

27. The plasma or serum fraction of claim 26, wherein the inoculant is a bacteria-bearing inoculant.

28. The plasma or serum fraction of claim 26, wherein the inoculant is a viral-bearing inoculant.

29. A plasma or serum fraction useful in the treatment and prevention of bacterial infection and related conditions, which fraction is derived a mammal exposed to an inoculant, and which fraction has been depleted of proteins or biological agents with a molecular weight greater than approximately 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 or 65 kD present in the unprocessed plasma or serum.

30. The plasma or serum fraction of claim 29, wherein the inoculant is a bacteria-bearing inoculant.

31. The plasma or serum fraction of claim 29, wherein the inoculant is a viral-bearing inoculant.

32. A method for treating or preventing a bacterial infection or related conditions by administering to a subject in need thereof a therapeutic amount of a plasma or serum fraction, which fraction is derived by exposing a mammal to an inoculant, and which fraction is depleted of one or more high molecular weight proteins or biological agents present in the unprocessed plasma or serum, either alone or in combination or alternation with another anti-bacterial agent or agent that treats a related condition.

33. The method of claim 32, wherein the subject is a human.

34. The method of claim 32, wherein the bacterial infection is a Gram-positive bacterial infection.

35. The method of claim 34, wherein the Gram-positive bacterial infection is selected from the group consisting of Staphylococcus infections, Streptococcus infections, Enterococcus infections and related conditions.

36. The method of claim 34, wherein the Gram-positive bacterial infection is Staphylococcus aureus.

37. The method of claim 34, wherein the Gram-positive bacterial infection is methicillin-resistant Staphylococcus aureus.

38. The method of claim 34, wherein the Gram-positive bacterial infection is Streptococcus pneumonia.

39. The method of claim 34, wherein the Gram-positive bacterial infection is penicillin resistant Streptococcus pneumonia.

40. The method of claim 34, wherein the Gram-positive bacterial infection is vancomycin-resistant Enterococcus.

41. The method of claim 34, wherein the infection is a Gram-negative bacterial infection selected from consisting of Salmonella, Shigella, Escherichia, Klebsiella, Enterobacter, Serratia, Proteus, Morganella, Providencia, Yersinia, Neisseria, Moraxella (Branhamella) and Acinetobacter infections and related conditions.

42. The method of claim 34, wherein the plasma or serum fraction is administered by subcutaneous injection.

43. The method of claim 34, wherein the inoculant is a viral-bearing inoculant.

44. The method of claim 34, wherein the inoculant is a bacteria-bearing inoculant.

45. The method of claim 44, wherein the bacterial-bearing inoculant is a Gram-positive-bearing bacterial inoculant or a Gram-negative bacterial-bearing inoculant.

46. The method of claim 34, wherein the plasma or serum fraction is depleted of immunoglobulin present in the unprocessed plasma or serum.

47. The method of claim 34, wherein the plasma or serum fraction is depleted of two or more high molecular weight proteins or biological agents present in the unprocessed plasma or serum.

48. The method of claim 47, wherein the plasma or serum fraction is depleted of immunoglobulin and albumin present in the unprocessed plasma or serum.

49. A method of preparing a plasma or serum fraction useful in the treatment or prevention of a bacterial infection or related condition, involving (a) exposing a mammal to an inoculant; (b) allowing time for the mammal to respond to the inoculant and to produce one or more beneficial biologic agents in the blood; and (c) obtaining the plasma or serum; (d) processing the plasma or serum to isolate the anti-bacterial activity from one or more high molecular weight proteins or biological agents present in the unprocessed plasma or serum.

50. The method of claim 49, wherein the mammal is an ungulate.

52. The method of claim 50, wherein the ungulate is a goat.

53. The method of claim 49, wherein the mammal is not susceptible to infection with the inoculant.

54. The method of claim 49, wherein the inoculant is a bacteria-bearing inoculant.

55. The method of claim 49, wherein the bacteria-bearing inoculant is a Gram-positive-bearing bacterial inoculant or a Gram-negative bacteria-bearing inoculant.

56. The method of claim 55, wherein the inoculant is a viral-bearing inoculant.

57. The method of claim 49, wherein the plasma or serum is processed to isolate the anti-bacterial activity by a fractionation method.

58. The method of claim 57, wherein the fractionation method is selected from the group consisting of fractional precipitation, dialysis and ultrafiltration, and/or chromatographic fractionation.

59. The method of claim 58, wherein the fractional precipitation is ammonium sulfate precipitation.

60. The method of claim 58, wherein the chromatographic fractionation is selected from the group of gel filtration chromatography, ion exchange chromatography and affinity chromatography.

61. The method of claim 57, wherein the fractionation method involves a single fractionation step.

62. The method of claim 57, wherein the fractionation method involves two or more fractionation steps.

63. The method of claim 62, wherein the two or more fractionation steps involve the same or different fractionation methods.

64. The method of claim 62, wherein the fractionation involves a first ammonium sulfate precipitation step and a second DEAE-column chromatography step.
